(19) United States
(12) Patent Application Publication
(10) Pub. No.: US 2023/0040485 A1
(43) Pub. Date: Feb. 9, 2023

(54) HYBRID POLYMERIC MATERIALS AND USES THEREOF

(71) Applicant: Allergan Pharmaceuticals International Limited, Dublin 17 (IE)

(72) Inventors: Anthony Steven WEISS, Sydney (AU); Suzanne Marie MITHIEUX, Sydney (AU); Ziyu WANG, Sydney (AU)

(21) Appl. No.: 17/757,693

(22) PCT Filed: Dec. 18, 2020
(86) PCT No.: PCT/IB2020/062153

§ 371 Date: Jun. 17, 2022

Related U.S. Application Data
(60) Provisional application No. 62/971,190, filed on Feb. 6, 2020.

(30) Foreign Application Priority Data
Dec. 18, 2019 (AU) 2019904795

(51) Int. Cl. A61L 27/26; A61L 27/56; D01D 1/02; D01D 5/00; D01F 4/00

(52) U.S. Cl. CPC A61L 27/26; A61L 27/56; D01D 1/02; D01D 5/003; D01D 5/0076; D01F 4/00; A61L 2430/20; D10B 2509/00

(57) ABSTRACT

Disclosed herein is a hybrid polymeric material comprising a tropoelastin and a copolymer of a polyol monomer and a polycarboxylic acid monomer. The hybrid polymeric material is suitable for use as a tissue scaffold.

### FIELD

The disclosure relates to hybrid polymeric materials that are suitable for use as a tissue scaffold.

### BACKGROUND

Elastin is an extracellular matrix protein found in many tissues and organs that require a degree of flexibility to function, such as skin, blood vessels, elastic ligaments, bladder, and lungs. Elastin is comprised of cross-linked tropoelastin monomers and plays a pivotal structural and biological role within the extracellular matrix.

An increasing number of approaches are designed to facilitate delivery of elastin to damaged or diseased tissues in order to provide a conducive environment for functional tissue regeneration. The compromised ability of adult cells to synthesize extensive organized elastin fiber networks makes such strategies vital. Tissue regenerative approaches that facilitate the generation of elastin in a form that mimics its composition, architecture, and function in native tissues are highly sought after. Therefore, forming organised elastin is key to the next generation of elastic tissue, because it converts regenerated tissue to a natural and functional state.

Synthetic implants are useful to repair or replace damaged tissue, such as at a wound site or for the replacement of blood vessel sections. Materials for such implants ideally are durable, compatible with the surrounding tissue and have mechanical properties that match the original tissue. The requirement for tissue matching compliance and durability is particularly critical in tissue engineered blood vessels (TEBVs) where incompatibility can lead to graft failure through aneurysm. Elastic fiber content and architecture can not only determine the mechanical properties of TEBVs but can also inhibit smooth muscle cell proliferation that leads to graft occlusion.

Accordingly, there is a need for developing materials for implants that facilitate improved formation of functional elastin upon implantation. There is also a need for developing materials that have suitable tissue matching compliance and/or durability for use in tissue scaffolds. Further, there is a need for developing materials that promote rapid endothelialisation, and/or reduce intimal hyperplasia. There is also a need for developing materials that promote connective tissue deposition, for example, that promote the synthesis and organisation of collagen and the organisation of cells, and/or that have a matched material degradation rate with tissue remodelling.

It is an aim of the present disclosure to at least partially satisfy at least one of the above needs.

### SUMMARY

The disclosure provides a new hybrid polymeric material that surprisingly and unexpectedly promotes elastin network formation on implantation. This hybrid polymeric material exhibits mechanical, structural, and/or biocompatibility properties that may be suitable as a scaffold for tissue regeneration.

According to a first aspect of the disclosure there is provided a hybrid polymeric material comprising: a tropoelastin; and a copolymer of a polyol monomer and a polycarboxylic acid monomer.

The following options may be used in conjunction with the first aspect, either individually or in any suitable combination.

The polyol monomer may be a triol. It may be, for example, glycerol.

The polycarboxylic acid monomer may be a dicarboxylic acid. It may be a linear C₄-C₂₀ dicarboxylic acid. It may be, for example, sebacic acid.

The hybrid polymeric material may comprise a copolymer of tropoelastin and poly(glycerol sebacate).

The mass ratio of the tropoelastin to the polyol-polycarboxylic acid copolymer may be from about 1:99 to about 99:1. In some embodiments, the mass ratio of the tropoelastin to the polyol-polycarboxylic acid copolymer is about 1:90, 1:80, 1:70, 1:60, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, or 90:1. In some embodiments, the mass ratio of the tropoelastin to the polyol-polycarboxylic acid copolymer is preferably from about 50:50 to about 70:30.

The hybrid polymeric material may comprise fibers. The fibers may have an average fiber width of from about 5 nm to about 10 μm. In some embodiments, the hybrid polymeric material may have an average fiber width of about 5 nm, 10 nm, 20 nm, 30 nm, 40 nm, 50 nm, 60 nm, 70 nm, 80 nm, 90 nm, 100 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 700 nm, 800 nm, 900 nm, 1 μm, 2 μm, 3 μm, 4 μm, 5 μm, 6 μm, 7 μm, 8 μm, 9 μm, or 10 μm. In certain embodiments, the hybrid polymeric material may, for example have an average fiber width of from about 200 nm to about 600 nm.

The hybrid polymeric material may have a porous structure. It may have an average pore size (e.g., diameter) of from about 0.05 μm to about 1000 μm. In embodiments, the hybrid polymeric material may have an average pore size of about 50 nm, 60 nm, 70 nm, 80 nm, 90 nm, 100 nm, 200 nm, 300 nm, 400 nm, 500 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, 1 μm, 1.1 μm, 1.2 μm, 1.3 μm, 1.4 μm, 1.5 μm, 1.6 μm, 1.7 μm, 1.8 μm, 1.9 μm, 2 μm, 3 μm, 4 μm, 5 μm, 6 μm, 7 μm, 8 μm, 9 μm, or 10 μm. In exemplary embodiments, the hybrid polymeric material has an average pore size of from about 0.6 μm to about 1.5 μm. In some embodiments, the hybrid polymeric material may have a percentage porosity of from about 30% to about 60%.

The tropoelastin may have at least about 70% to about 100% sequence identity with the amino acid sequence of a human tropoelastin isoform across at least 50 consecutive amino acids. In certain embodiments, the tropoelastin of the disclosure has at least about 70%, 75%, 80%, 85%, 90%, 95%, or 100% sequence identity with the amino acid sequence of a human tropoelastin isoform across at least 50 consecutive amino acids. In some embodiments, the tropoelastin may have the sequence of a human tropoelastin isoform.

In certain embodiments the hybrid polymeric material comprises a copolymer of tropoelastin and poly(glycerol sebacate), wherein the mass ratio of the tropoelastin to the poly(glycerol sebacate) is from about 50:50 to about 70:30; the hybrid polymeric material comprises fibers having an average fiber width of from about 200 nm to about 600 nm; and the hybrid polymeric material has a porous structure, having an average pore size of from about 0.6 μm to about 1.5 μm, and a percentage porosity of from about 30% to about 60%.

According to a second aspect of the disclosure there is provided a tissue scaffold comprising the hybrid polymeric material according to the first aspect.

The following options may be used in conjunction with the second aspect, either individually or in any suitable combination.

The tissue scaffold may have a Young's modulus of from about 0.01 MPa to about 80 MPa. In some embodiments, the tissue scaffold may have a Young's modulus of about 0.01 MPa, 0.01 MPa, 0.02 MPa, 0.03 MPa, 0.04 MPa, 0.05 MPa, 0.06 MPa, 0.07 MPa, 0.08 MPa, 0.09 MPa, 0.1 MPa, 0.2 MPa, 0.3 MPa, 0.4 MPa, 0.5 MPa, 0.6 MPa, 0.7 MPa, 0.8 MPa, 0.9 MPa, 1.0 MPa, 2.0 MPa, 3.0 MPa, 4.0 MPa, 5.0 MPa, 6.0 MPa, 7.0 MPa, 8.0 MPa, 9.0 MPa, 10 MPa, 20 MPa, 30 MPa, 40 MPa, 50 MPa, 60 MPa, 70 MPa, 80 MPa, 90 MPa, or 100 MPa. In certain embodiments, the tissue scaffold may have a Young's modulus of from about 1 MPa to about 30 MPa.

The tissue scaffold may have an ultimate tensile strength of from about 0.01 MPa to about 80 MPa. In some embodiments, the tissue scaffold may have an ultimate tensile strength of about 0.01 MPa, 0.01 MPa, 0.02 MPa, 0.03 MPa, 0.04 MPa, 0.05 MPa, 0.06 MPa, 0.07 MPa, 0.08 MPa, 0.09 MPa, 0.1 MPa, 0.2 MPa, 0.3 MPa, 0.4 MPa, 0.5 MPa, 0.6 MPa, 0.7 MPa, 0.8 MPa, 0.9 MPa, 1.0 MPa, 1.1 MPa, 1.2 MPa, 1.5 MPa, 2.0 MPa, 3.0 MPa, 4.0 MPa, 5.0 MPa, 6.0 MPa, 7.0 MPa, 8.0 MPa, 9.0 MPa, 10 MPa, 11.0 MPa, 12.0 MPa, 15.0 MPa, 20 MPa, 21 MPa, 22 MPa, 25 MPa, 30 MPa, 35 MPa, 40 MPa, 50 MPa, 60 MPa, 70 MPa, 80 MPa, 90 MPa, or 100 MPa. In certain embodiments, the tissue scaffold may have an ultimate tensile strength of from about 2 MPa to about 10 MPa.

The tissue scaffold of the disclosure may have a percentage elongation at failure of from about 30% to about 400%. In embodiments, the tissue scaffold may have a percentage elongation at failure of from about 10%, 20%, 30%, 35%, 40%, 45%, 50%, 55%, 60%, 70%, 75%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, or 400%. In certain embodiments, the tissue scaffold may have a percentage elongation at failure of from about 40% to about 110%.

The tissue scaffold may lose less than about 40% of its mass when incubated at 37° C. in PBS for 1 week. In embodiments, the tissue scaffold may lose less than about 40%, 35%, 30%, 25%, 20%, 15%, 10%, 9%, 8%, or 7% of its mass when incubated at 37° C. in PBS for 1 week.

The tissue scaffold of the second aspect may be made of the hybrid polymeric material of the first aspect. In certain embodiments, the hybrid polymeric material of the first aspect may be used in the tissue scaffold of the second aspect.

According to a third aspect of the disclosure there is provided a method for producing a hybrid polymeric material, said method comprising the following steps:
(A) providing a mixture comprising:
   a tropoelastin; and
   a copolymer of a polyol monomer and a polycarboxylic acid monomer; and
(B) heating the mixture to form the hybrid polymeric material;
   wherein the tropoelastin, polyol monomer, and polycarboxylic acid monomer are as defined according to the first aspect.

The following options may be used in conjunction with the third aspect, either individually or in any suitable combination.

The heating may be at a temperature of from about 50° C. to about 220° C. In some embodiments, the method for producing a hybrid polymeric material according to the disclosure comprises heating the mixture at a temperature of from about 50° C., 60° C., 70° C., 80° C., 90° C., 100° C., 110° C., 120° C., 130° C., 140° C., 150° C., 160° C., 170° C., 180° C., 190° C., 200° C., 210° C., or 220° C. In some embodiments the method for producing a hybrid polymeric material according to the disclosure comprises heating the mixture at a temperature of about 160° C.

The heating may be for a duration of from about 10 minutes to about 24 hours. In certain embodiments, the method comprises heating the mixture for a duration of about 10 minutes, 20 minutes, 30 minutes, 40 minutes, 50 minutes, 1 hour, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.

The method may be performed at a pressure of about 1 atmosphere.

The mixture may comprise a solvent, and the method may further comprise a step of removing or reducing the amount of solvent prior to step (B). The solvent may be a polar organic solvent, having a boiling point below 80° C. In some embodiments, the polar organic solvent has a boiling point below 50° C., 60° C., or 70° C. The solvent may be, for example, hexafluoro-2-propanol.

The method may comprise a step of electrospinning the mixture. The mixture may be electrospun onto a polytetrafluoroethylene-coated mandrel.

In certain embodiments the methods do not comprise a step of heating a solution of tropoelastin.

In certain embodiments the method comprises the following steps: providing a mixture comprising tropoelastin, poly(glycerol sebacate), and hexafluoro-2-propanol, wherein the mass ratio of the tropoelastin to the poly(glycerol sebacate) is from about 50:50 to about 70:30; electrospinning the mixture under conditions to remove or reduce the amount of the hexafluoro-2-propanol; heating the mixture at about 160° C. for greater than 2 hours to form the hybrid polymeric material; wherein the hybrid polymeric material comprises fibers having an average fiber width of from about 200 nm to about 600 nm; and the hybrid polymeric material has a porous structure, having an average pore size of from about 0.6 μm to about 1.5 μm, and a percentage porosity of from about 30% to about 60%.

In the method of the third aspect may produce the hybrid polymeric material of the first aspect. The hybrid polymeric material of the first aspect may be produced using the method of the third aspect.

The method of the third aspect may produce the tissue scaffold of the second aspect. The tissue scaffold of the second aspect may be produced using the method of the third aspect.

According to a fourth aspect of the disclosure there is provided a tissue scaffold made according to the method of the third aspect.

The following options may be used in conjunction with the fourth aspect, either individually or in any suitable combination.

The tissue scaffold may be a vascular graft, a heart valve, nerve guide, surgical patch, or a wound-healing scaffold.

The tissue scaffold of the fourth aspect may be produced using the method of the third aspect. The method of the third aspect may produce the tissue scaffold of the fourth aspect.

The tissue scaffold of the fourth aspect may be made of the hybrid polymeric material of the first aspect. The hybrid polymeric material of the first aspect may be used in the tissue scaffold of the fourth aspect.

According to a fifth aspect of the disclosure there is provided the use of the hybrid polymeric material according to the first aspect in the manufacture of a tissue scaffold.

The use of the fifth aspect may use the method according to the third aspect. The method of the third aspect may be used in the use of the fifth aspect.

The use of the fifth aspect may produce the tissue scaffold of the second or fourth aspect. The tissue scaffold of the second or fourth aspect may be produced according to the use of the fifth aspect.

According to a sixth aspect of the disclosure there is provided a method for regenerating tissue in a subject in need thereof, comprising implanting or applying the tissue scaffold according to the second or fourth aspect in or on the subject.

The method of the sixth aspect may use the tissue scaffold of the second or fourth aspect. The tissue scaffold of the second or fourth aspect may be used in the method of the sixth aspect.

### BRIEF DESCRIPTION OF DRAWINGS

FIG. 1 shows a schematic depiction of the fabrication of example scaffolds using electrospinning and solvent casting methods.

FIG. 2 shows macroscopic and scanning electron microscope (SEM) images of example electrospun tropoelastin-poly(glycerol sebacate (TE-PGS) scaffolds. The scale bars for the unheated and heated scaffold images are 1 cm in length. The scale bars for the SEM images are 5 μm in length.

FIGS. 3A-C show characterization of (A) fiber width, (B) porosity, and (C) pore size of electrospun TE-PGS scaffolds.

FIG. 4 shows the 3D structure of TE-PGS scaffolds using autofluorescence. The scale bars are 10 μm in length.

FIG. 5 shows FTIR-ATR spectra of electrospun TE-PGS scaffolds and a 100:0 TE (HeaTro) scaffold sample, for: (A) unheated, and (B) heated scaffolds.

FIG. 6 shows swelling properties of electrospun TE-PGS scaffolds and a 100:0 (HeaTro) scaffold.

FIG. 7 shows stress-strain curves of electrospun TE-PGS scaffolds.

FIG. 8 shows mass degradation of electrospun TE-PGS scaffolds over 6 weeks.

FIGS. 9A-C show proliferation of (A) human dermal fibroblasts (HDFs), (B) human umbilical vein endothelial cells (HUVECs) and (C) human coronary artery smooth muscle cells (HCASMCs) on solvent cast PGS (SC-PGS) and electrospun TE:PGS (ES-50:50 and ES-70:30) scaffolds for 1, 3 and 7 days. ES-50:50=electrospun TE:PGS-50:50. ES-70:30=electrospun TE:PGS-70:30.

FIG. 10 shows F-actin staining of HUVECs cultured on PGS (SC-PGS) and TE:PGS (ES-50:50 and ES-70:30) scaffolds at 1, 3 and 7 days post-seeding. The scale bars are 50 μm in length.

FIG. 11 shows F-actin staining of HCASMCs cultured on solvent cast PGS (SC-PGS) and electrospun TE:PGS (ES-50:50 and ES-70:30) scaffolds for 1, 3 and 7 days. The scale bars are 50 μm in length.

FIGS. 12A-C show data for subcutaneously implanted TE-PGS in mice. FIG. 12A shows hematoxylin and eosin (H&E) and Masson's trichrome histology staining for PGS scaffolds and TE-PGS scaffolds subcutaneously cultured in mice for 2 and 4 weeks. The scale bars are 100 μm in length. FIG. 12B shows normalized tissue area in the tissue surrounding the implant. FIG. 12C shows total cell number in the tissue surrounding the implant.

FIG. 13 shows a schematic depiction of an example electrospinning fabrication process for a TE-PGS vascular graft.

FIGS. 14A-G show electron micrographs of electrospun TE-PGS vascular graft appearance and morphology. FIG. 14A shows a gross image of an ES-50:50 vascular graft before 1502 and after 1504 heating. FIG. 14B shows vascular graft cross-sectional morphology. FIG. 14C shows vascular wall lumen surface morphology. FIG. 14D shows lumen surface morphology before heating. FIG. 14E shows lumen surface morphology after heating. FIG. 14F shows vascular outer wall surface morphology. FIG. 14G shows outer wall surface morphology before heating. FIG. 14H shows outer wall surface morphology after heating.

FIG. 15 shows multi-photon microscopy of 3D structures within TE-PGS scaffolds of the compositions indicated at the top of each column. TE is visualized through its autofluorescence (top row) and the PGS component is stained by Rhodamine 6G (middle row). A merged image of TE and PGD is shown in the bottom row. The scale bar is 20 μm in length.

FIG. 16 shows proliferation assays and fluorescence microscopy of HDFs on SC-PGS film and electropsun TE-PGS films of the indicated composition. The plots at top show HDF proliferation data at 1, 3, and 7 days after seeding onto the films. The micrographs at bottom show F-actin (diffuse cytoplasmic) and nuclear (punctate) staining of HDFs at 7 days after seeding. Scale bar=100 μm.

FIGS. 17A-D show data for vascular endothelial cell proliferation and function after culture on TE-PGS scaffolds. FIG. 17A shows HUVEC proliferation profiles on SC-PGS film and electropsun TE-PGS films at 1, 3, and 7 days after seeding. FIG. 17B shows shows F-actin and DAPI staining of HUVECs cultured on solvent cast PGS (SC-PGS) and electrospun TE:PGS (ES-50:50 and ES-70:30) scaffolds at day 1 and day 7 after seeding (scale bar 100 μm). FIG. 17C shows gene expression of vascular-related functions in HUVECs cultured on solvent cast PGS (SC-PGS) and electrospun TE:PGS (ES-50:50 and ES-70:30) scaffolds at day 1 and day 7 after seeding. FIG. 17D shows confocal fluorescence images of anti-VE-Cadherin stained (top), anti-eNOS stained (middle), and anti-vWF stained (bottom) in HUVECs cultured on solvent cast PGS (SC-PGS) and electrospun TE:PGS (ES-50:50 and ES-70:30) scaffolds at day 7 after seeding (scale bar 25 μm).

FIG. 18: shows SEM images of thick electrospun vascular grafts with TE:PGS ratios of 50:50 and 70:30.

FIGS. 19A-F show data on implanted ES-50:50 grafts in mouse aorta. FIG. 19A shows histology of haematoxylin and eosin (HE, top three rows), Picrosirius red (PSR, middle three rows), Verhoeff-Van Gieson (VVG, bottom three rows) for native mouse aorta, graft proximal, and graft middle. FIG. 19B shows elastin autofluorescence in native mouse aorta, graft proximal, and graft middle. FIG. 19C shows lumen size for graft proximal, graft middle, and native aorta. FIG. 19D shows wall thickness for graft proximal, graft middle, and native aorta. FIG. 19E shows elastic fiber fraction for graft proximal, graft middle, and native aorta. FIG. 19F shows elastic fiber thickness for graft proximal, graft middle, and native aorta.

### DETAILED DESCRIPTION

### Definitions

PBS: phosphate-buffered saline; TE: tropoelastin; PGS: poly(glycerol sebacate); FTIR-ATR: Fourier-transform infrared attenuated total reflectance; SEM: scanning electron microscopy; HDF: human dermal fibroblasts; HUVEC: human umbilical vein endothelial cells; HCASMC: human coronary artery smooth muscle cells; SC: solvent cast; ES: electrospun; HFP: hexafluoro-2-propanol; PTFE: polytetrafluoroethylene.

### Hybrid Polymeric Materials

Disclosed herein are hybrid polymeric materials that can comprise a tropoelastin. Optionally, some embodiments may comprise a copolymer of a polyol monomer. Further optionally, some embodiments may comprise a polycarboxylic acid monomer. Such embodiments and uses thereof are also disclosed herein.

### Polyol Monomer

The polyol monomer may have from about 2 to about 10 hydroxyl groups. It may be, for example, a diol, triol, tetraol, pentaol, hexaol, or heptaol. It may be a low molecular weight polyol (i.e. having a molecular weight below 900 Daltons). It may be selected from the group consisting of glycerol, ethylene glycols, xylitol, pentaerythritol, and combinations thereof. It may be a sugar, or sugar derivate. It may be, for example, a triol, such as glycerol.

### Polycarboxylic Acid Monomer

The polycarboxylic acid monomer may have from about 2 to about 10 carboxylic acid groups. It may be, for example, a dicarboxylic acid, tricarboxylic acid, tetracarboxylic acid, pentacarboxylic acid, hexacarboxylic acid, or heptacarboxylic acid. It may be a low molecular weight polycarboxylic acid (i.e. having a molecular weight below 900 Daltons). It may be selected form the group consisting of oxalic acid, malonic acid, succinic acid, glutaric acid, adipic acid, pimelic acid, suberic acid, azelaic acid, sebacic acid, undecanedioic acid, dodecanedioic acid, tridecanedioic acid, hexadecanedioic acid, docosanedioic acid, citric acid, propane-1,2,3-tricarboxylic acid, isocitric acid, aconitic acid, and combinations thereof. It may be, for example, a dicarboxylic acid. It may be a linear or branched C₄-C₂₀ di-, tri-, or tetra-carboxylic acid. It may be a linear or branched C₄-C₂₀ dicarboxylic acid. It may be, for example, a linear C₄-C₂₀ dicarboxylic acid, such as sebacic acid.

### Tropoelastin

Tropoelastin is a monomeric protein encoded by the elastin genomic sequence (or gene). Tropoelastin is approximately 60-70 kDa in size. There are about 36 small domains in tropoelastin and each weigh about 2 kDa. Within the exons, there are alternating hydrophobic domains rich in non-polar amino acids such as glycine, valine, proline, isoleucine and leucine (which domains often occur in repeats of three to six peptides such as GVGVP (SEQ ID NO: 1), GGVP (SEQ ID NO: 2) and GVGVAP (SEQ ID NO: 3), and hydrophilic domains rich in lysine and alanine. The hydrophilic domains often consist of stretches of lysine separated by two or three alanine residues such as AAAKAAKAA (SEQ ID NO: 4). Additionally, tropoelastin ends with a hydrophilic carboxy-terminal sequence containing its only two cysteine residues.

In certain embodiments the tropoelastin that is used in the hybrid polymeric material disclosed herein includes both hydrophilic and hydrophobic domains. Hydrophilic domains contribute to elastic function (by, for example, binding to water). They also contribute to a wider variety of biological functions including binding to cells and to the extra-cellular matrix. The hydrophobic domains are believed to be important for providing elasticity.

Some examples of amino acid sequences that may be present in the tropoelastin used in the hybrid polymeric material disclosed herein are as follows:

(SEQ ID NO: 5)
GGVPGAIPGGVPGGVFYP,
(SEQ ID NO: 6)
GVGLPGVYP,
(SEQ ID NO: 7)
GVPLGYP,
(SEQ ID NO: 8)
PYTTGKLPYGYGP,
(SEQ ID NO: 9)
GGVAGAAGKAGYP,
(SEQ ID NO: 10)
TYGVGAGGFP,
(SEQ ID NO: 11)
KPLKP,
(SEQ ID NO: 12)
ADAAAAYKAAKA,
(SEQ ID NO: 13)
GAGVKPGKV,
(SEQ ID NO: 14)
GAGVKPGKV,
(SEQ ID NO: 15)
TGAGVKPKA,
(SEQ ID NO: 16)
QIKAPKL,
(SEQ ID NO: 17)
VAPGVG,
(SEQ ID NO: 18)
VPGVG,
(SEQ ID NO: 19)
AAAAAAAKAAAK,
(SEQ ID NO: 20)
AAAAAAAAAAKAAKYGAAAGLV,
(SEQ ID NO: 21)
EAAAKAAAKAAKYGAR,
(SEQ ID NO: 22)
EAQAAAAAKAAKYGVGT,
(SEQ ID NO: 23)
AAAAAKAAAKAAQFGLV
(SEQ ID NO: 24)
GGVAAAAKSAAKVAAKAQLRAAAGLGAGI,
(SEQ ID NO: 25)
GALAAAKAAKYGAAV,
(SEQ ID NO: 26)
AAAAAAAKAAAKAA,
(SEQ ID NO: 27)
AAAAKAAKYGAA,
and/or
(SEQ ID NO: 28)
CLGKACGRKRK.

The tropoelastin for use in the hybrid polymeric material disclosed herein may, in certain embodiments, include or consist of, any one of the above described sequences.

In one embodiment the tropoelastin for use in the hybrid polymeric material disclosed herein includes or consists of a sequence shown below: VXPGVG (SEQ ID NO: 29) where X is any amino acid residue or no residue, ZXPGZG (SEQ ID NO: 30) wherein Z is an aliphatic residue, VXP(I/L/V)V(I/L/V) wherein (I/L/V) is isoleucine, leucine or valine.

In one embodiment, the tropoelastin for use in the hybrid polymeric material disclosed herein contains hydrophilic and hydrophobic domains of tropoelastin. Other suitable tropoelastin sequences are known in the art and include CAA33627 (Homo sapiens), P15502 (Homo sapiens), AAA42271 (Rattus norvegicus), AAA42272 5 (Rattus norvegicus), AAA42268 (Rattus norvegicus), AAA42269 (Rattus norvegicus), AAA80155 (Mus musculus), AAA49082 (Gallus gallus), P04985 (Bos taurus), ABF82224 (Danio rerio), ABF82222 (Xenopus tropicalis) and P11547 (Ovis aries). In a preferred embodiment, the tropoelastin for use in the hybrid polymeric material disclosed herein is derived from human tropoelastin. As stated herein, the hybrid polymeric material disclosed herein also includes variants, for example species variants, or polymorphic variants, of tropoelastin. The tropoelastin for use in the hybrid polymeric material disclosed herein may be obtained from recombinant sources. They can also be extracted from natural sources or synthesised (by, for example, solid-phase synthesis techniques). Tropoelastin is also commercially available.

There are a number of isoforms of tropoelastin and therefore the exact number of amino acids that make up the tropoelastin polypeptide will vary. The hybrid polymeric material disclosed herein also includes variants of tropoelastin, for example species variants or polymorphic variants. The hybrid polymeric material disclosed herein is intended to cover all functionally-active variants of tropoelastin that exhibit the same activity (i.e. biocompatibility and elasticity). This also includes apo- and holo-forms of tropoelastin, post-translationally modified forms, as well as glycosylated or de-glycosylated derivatives. Such functionally-active fragments and variants include, for example, those having conservative amino acid substitutions.

In one embodiment, the tropoelastin for use in the hybrid polymeric material disclosed herein is the SHELδ26A tropoelastin analogue (WO 1999/03886). The amino acid sequence of SHELδ26A

(SEQ ID NO: 31)
GGVPGAIPGGVPGGVFYPGAGLGALGGGALGPGGK
PLKPVPGGLAGAGLGAGLGAFPAVTFPGALVPGGV
ADAAAAYKAAKAGAGLGGVPGVGGLGVSAGAVVPQ
PGAGVKPGKVPGVGLPGVYPGGVLPGARFPGVGVL
PGVPTGAGVKPKAPGVGGAFAGIPGVGPFGGPQPG
VPLGYPIKAPKLPGGYGLPYTTGKLPYGYGPGGVA
GAAGKAGYPTGTGVGPQAAAAAAAKAAAKFGAGAA
GVLPGVGGAGVPGVPGAIPGIGGIAGVGTPAAAAA
AAAAAKAAKYGAAAGLVPGGPGFGPGVVGVPGAGV
PGVGVPGAGIPVVPGAGIPGAAVPGVVSPEAAAKA
AAKAAKYGARPGVGVGGIPTYGVGAGGFPGFGVGV
GGIPGVAGVPSVGGVPGVGGVPGVGISPEAQAAAA
AKAAKYGVGTPAAAAAKAAAKAAQFGLVPGVGVAP
GVGVAPGVGVAPGVGLAPGVGVAPGVGVAPGVGVA
PGIGPGGVAAAAKSAAKVAAKAQLRAAAGLGAGIP
GLGVGVGVPGLGVGAGVPGLGVGAGVPGFGAVPGA
LAAAKAAKYGAAVPGVLGGLGALGGVGIPGGVVGA
GPAAAAAAAKAAAKAAQFGLVGAAGLGGLGVGGLG
VPGVGGLGGIPPAAAAKAAKYGAAGLGGVLGGAGQ
FPLGGVAARPGFGLSPIFPGGACLGKACGRKRK.

In another embodiment, the tropoelastin for use in the hybrid polymeric material disclosed herein is the SHEL isoform (WO 1994/14958; included by reference in its entirety herein): SMGGVPGAIPGGVPGGVFYPGAGLGALGGGALGPGGKPLKPVPGGLAGAGLGAGLGA FPAVTFPGALVPGGVADAAAAYKAAKAGAGLGGVPGVGGLGVSAGAVVPQPGAGVK PGKVPGVGLPGVYPGGVLPGARFPGVGVLPGVPTGAGVKPKAPGVGGAFAGIPGVGPF GGPQPGVPLGYPIKAPKLPGGYGLPYTTGKLPYGYGPGGVAGAAGKAGYPTGTGVGPQ AAAAAAAKAAAKFGAGAAGVLPGVGGAGVPGVPGAIPGIGGIAGVGTPAAAAAAAAA AKAAKYGAAAGLVPGGPGFGPGVVGVPGAGVPGVGVPGAGIPVVPGAGIPGAAVPGV VSPEAAAKAAAKAAKYGARPGVGVGGIPTYGVGAGGFPGFGVGVGGIPGVAGVPSVG GVPGVGGVPGVGISPEAQAAAAAKAAKYGVGTPAAAAAKAAAKAAQFGLVPGVGVA PGVGVAPGVGVAPGVGLAPGVGVAPGVGVAPGVGVAPGIGPGGVAAAAKSAAKVAA KAQLRAAAGLGAGIPGLGVGVGVPGLGVGAGVPGLGVGAGVPGFGAGADEGVRRSLS PELREGDPSSSQHLPSTPSSPRVPGALAAAKAAKYGAAVPGVLGGLGALGGVGIPGGVV GAGPAAAAAAAKAAAKAAQFGLVGAAGLGGLGVGGLGVPGVGGLGGIPPAAAAKAA KYGAAGLGGVLGGAGQFPLGGVAARPGFGLSPIFPGGACLGKACGRKRK) ((SEQ ID NO: 32) or a protease resistant derivative of the SHEL or SHELδ26A isoforms (WO 2000/04043; included by reference in its entirety herein). As described in WO 2000/04043, the protein sequences of tropoelastin described may have a mutated sequence that leads to a reduced or eliminated susceptibility to digestion by proteolysis. Without being limiting, the tropoelastin amino acid sequence has a reduced or eliminated susceptibility to serine proteases, thrombin, kallikrein, metalloproteases, gelatinase A, gelatinase B, serum proteins, trypsin or elastase, for example. In an embodiment, the tropoelastin comprises a SHELδ26A isoform: GGVPGAIPGGVPGGVFYPGAGLGALGGGALGPGGKPLKPVPGGLAGAGLGAGLGAFPA VTFPGALVPGGVADAAAAYKAAKAGAGLGGVPGVGGLGVSAGAVVPQPGAGVKPGK VPGVGLPGVYPGGVLPGARFPGVGVLPGVPTGAGVKPKAPGVGGAFAGIPGVGPFGGP QPGVPLGYPIKAPKLPGGYGLPYTTGKLPYGYGPGGVAGAAGKAGYPTGTGVGPQAAA AAAAKAAAKFGAGAAGVLPGVGGAGVPGVPGAIPGIGGIAGVGTPAAAAAAAAAAKA AKYGAAAGLVPGGPGFGPGVVGVPGAGVPGVGVPGAGIPVVPGAGIPGAAVPGVVSPE AAAKAAAKAAKYGARPGVGVGGIPTYGVGAGGFPGFGVGVGGIPGVAGVPSVGGVPG VGGVPGVGISPEAQAAAAAKAAKYGVGTPAAAAAKAAAKAAQFGLVPGVGVAPGVG VAPGVGVAPGVGLAPGVGVAPGVGVAPGVGVAPGIGPGGVAAAAKSAAKVAAKAQL RAAAGLGAGIPGLGVGVGVPGLGVGAGVPGLGVGAGVPGFGAVPGALAAAKAAKYG AAVPGVLGGLGALGGVGIPGGVVGAGPAAAAAAAKAAAKAAQFGLVGAAGLGGLGV GGLGVPGVGGLGGIPPAAAAKAAKYGAAGLGGVLGGAGQFPLGGVAARPGFGLSPIFP GGACLGKACGRKRK) (SEQ ID NO: 33). In some embodiments, the tropoelastin comprises a SHELδ mod isoform:

(SEQ ID NO: 34)
GGVPGAVPGGVPGGVFYPGAGFGAVPGGVADAAAA
YKAAKAGAGLGGVPGVGGLGVSAGAVVPQPGAGVK
PGKVPGVGLPGVYPGFGAVPGARFPGVGVLPGVPT
GAGVKPKAPGVGGAFAGIPGVGPFGGPQPGVPLGY
PIKAPKLPGGYGLPYTTGKLPYGYGPGGVAGAAGK
AGYPTGTGVGPQAAAAAAAKAAAKFGAGAAGFGAV
PGVGGAGVPGVPGAIPGIGGIAGVGTPAAAAAAAA
AAKAAKYGAAAGLVPGGPGFGPGVVGVPGFGAVPG
VGVPGAGIPVVPGAGIPGAAGFGAVSPEAAAKAAA
KAAKYGARPGVGVGGIPTYGVGAGGFPGFGVGVGG
IPGVAGVPSVGGVPGVGGVPGVGISPEAQAAAAAK
AAKYGVGTPAAAAAKAAAKAAQFGLVPGVGVAPGV
GVAPGVGVAPGVGLAPGVGVAPGVGVAPGVGVAPG
IGPGGVAAAAKSAAKVAAKAQLRAAAGLGAGIPGL
GVGVGVPGLGVGAGVPGLGVGAGVPGFGAVPGALA
AAKAAKYGAVPGVLGGLGALGGVGIPGGVVGAGPA
AAAAAAKAAAKAAQFGLVGAAGLGGLGVGGLGVPG
VGGLGGIPPAAAAKAAKYGAAGLGGVLGGAGQFPL
GGVAARPGFGLSPIFPGGACLGKACGRKRK).

In one embodiment, the tropoelastin has the sequence of a human tropoelastin isoform. The term “functionally-active” in relation to a fragment or variant of tropoelastin means the fragment or variant (such as an analogue, derivative or mutant) that is capable of forming an elastic material, as discussed further below. Such variants include naturally-occurring variants and non-naturally occurring variants. Additions, deletions, substitutions and derivatizations of one or more of the amino acids are contemplated so long as the modifications do not result in loss of functional activity of the fragment or variant. A functionally-active fragment can be easily determined by shortening the amino acid sequence, for example using an exopeptidase, or by synthesizing amino acid sequences of shorter length, and then testing for elastic material formation ability such as by methods described in WO2014/089610. Where non-natural variations occur, the fragment may be called a peptidomimetic, which are also within the scope of the disclosure. For example, synthetic amino acids and their analogues may be substituted for one or more of the native amino acids providing construct-forming activity as described in WO2014/089610. A “peptidomimetic” is a synthetic chemical compound that has substantially the same structure and/or functional characteristics of a tropoelastin for use in the hybrid polymeric material disclosed herein. A peptidomimetic generally contains at least one residue that is not naturally synthesized. Non-natural components of peptidomimetic compounds may be according to one or more of: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, polyproline turn, beta sheet, alpha helix conformation, and the like. Peptidomimetics can be synthesized using a variety of procedures and methodologies described in the scientific and patent literature.

The functionally-active fragment may be about 100 amino acids in length. Generally, the shortest fragment for use in the hybrid polymeric material disclosed herein will be about 10 amino acids in length. Therefore, the fragment may be between about 10 and about 100 amino acids in length.

In certain embodiments, the functionally-active fragment or variant has at least approximately 60% identity to a peptide such as described above, more preferably at least approximately 65%, 66%, 67%, 68%, 69%, 70%, 71%, 72%, 73%, 74%, 75%, 76%, 77%, 78%, 79%, 80%, 81%, 82%, 83%, 84% or 85% identity, even more preferably 90% identity, even more preferably at least approximately 95%, 96%, 97%, 98%, 99% or 100% identity. The functionally-active fragment or variant may correspond to, or have identity with, a contiguous sequence of amino acids from the tropoelastin, however it is also contemplated that a functionally-active fragment corresponds to, or has identity with, sequences of amino acids that are clustered spatially in the three-dimensional structure of the tropoelastin.

Such functionally-active fragments and variants include, for example, those having conservative amino acid substitutions. The term “conservative amino acid substitutions” refers to the substitution of an amino acid by another one of the same class, the classes being as follows:

Non-polar: Ala, Val, Leu, lie, Pro, Met, Phe, Trp; Uncharged polar: Gly, Ser, Thr, Cys, Tyr, Asn, Gln; Acidic: Asp, Glu; Basic: Lys, Arg, His. Other conservative amino acid substitutions may also be made as follows: Aromatic: Phe, Tyr, His; Proton Donor: Asn, Gln, Lys, Arg, His, Trp; Proton Acceptor: Glu, Asp, Thr, Ser, Tyr, Asn, Gln.

In one embodiment, the tropoelastin has a sequence that has at least 90% sequence identity with the amino acid sequence of human tropoelastin across at least 50 consecutive amino acids. In one embodiment, the tropoelastin has a sequence that has at least 80% sequence identity with the sequence of human tropoelastin across a consecutive amino acid sequence consisting of VPGVG (SEQ ID NO: 35).

One type of tropoelastin may be used in the hybrid polymeric material disclosed herein, or combinations of different tropoelastin may be used. For example, the combination of tropoelastin can include 1, 2, 3, 4, 5, 6, 7, 9, 10, or more different types of tropoelastin. In another embodiment, at least 2, at least 3, at least 4, at least 5, at least 6, at least 7, at least 8, at least 9, or at least 10 or more different tropoelastin types can be used. In another embodiment, 1 or more, 2 or more, 3 or more, 4 or more, 5 or more, 6 or more, 7 or more, 8 or more, 9 or more, or 10 or more different types of tropoelastin can be used.

In addition, in other embodiments, the tropoelastin can be any number or combination of human and/or non-human (e.g. primate, bovine, equine, sheep, goat, pig, dog, cat, or rodent) tropoelastin. Further, it will be appreciated that varying the ratio and/or identity of each of the tropoelastin types present in a combination can generate tropoelastin-based hydrogels with desired elasticity, tensile strength, and shapeability, and that the strength, elasticity, and other physical and biochemical behaviour of tropoelastin polymers can therefore be varied, and possibly controlled, by incorporating various polymorphic forms of tropoelastin into polymeric scaffolds. In addition, the ratio and/or identity of each of the tropoelastin types present in a combination can be varied so as to match the tropoelastin present in the tissue being repaired, replaced, or regenerated.

Recombinant forms of tropoelastin can be produced as shown in WO 1999/03886 for use in the hybrid polymeric material disclosed herein. These sequences are:

(SEQ ID NO: 36)
SMGGVPGAIPGGVPGGVFYPGAGLGALGGGALGPG
GKPLKPVPGGLAGAGLGAGLGAFPAVTFPGALVPG
GVADAAAAYKAAKAGAGLGGVPGVGGLGVSAGAVV
PQPGAGVKPGKVPGVGLPGVYPGGVLPGARFPGVG
VLPGVPTGAGVKPKAPGVGGAFAGIPGVGPFGGPQ
PGVPLGYPIKAPKLPGGYGLPYTTGKLPYGYGPGG
VAGAAGKAGYPTGTGVGPQAAAAAAAKAAAKFGAG
AAGVLPGVGGAGVPGVPGAIPGIGGIAGVGTPAAA
AAAAAAAKAAKYGAAAGLVPGGPGFGPGVVGVPGA
GVPGVGVPGAGIPVVPGAGIPGAAVPGVVSPEAAA
KAAAKAAKYGARPGVGVGGIPTYGVGAGGFPGFGV
GVGGIPGVAGVPSVGGVPGVGGVPGVGISPEAQAA
AAAKAAKYGVGTPAAAAAKAAAKAAQFGLVPGVGV
APGVGVAPGVGVAPGVGLAPGVGVAPGVGVAPGVG
VAPGIGPGGVAAAAKSAAKVAAKAQLRAAAGLGAG
IPGLGVGVGVPGLGVGAGVPGLGVGAGVPGFGAGA
DEGVRRSLSPELREGDPSSSQHLPSTPSSPRVPGA
LAAAKAAKYGAAVPGVLGGLGALGVGIPGGVVGAG
PAAAAAAAKAAAKAAQFGLVGAAGLGGLGVGGLGV
PGVGGLGGIPPAAAAKAAKYGAAGLGGVLGGAGQF
PLGGVAARPGFGLSPIFPGGACLGKACGRKRK;
(SEQ ID NO: 37)
GGVPGAIPGGVPGGVFYPGAGLGALGGGALGPGGK
PLKPVPGGLAGAGLGAGLGAFPAVTFPGALVPGGV
ADAAAAYKAAKAGAGLGGVPGVGGLGVSAGAVVPQ
PGAGVKPGKVPGVGLPGVYPGGVLPGARFPGVGVL
PGVPTGAGVKPKAPGVGGAFAGIPGVGPFGGPQPG
VPLGYPIKAPKLPGGYGLPYTTGKLPYGYGPGGVA
GAAGKAGYPTGTGVGPQAAAAAAAKAAAKFGAGAA
GVLPGVGGAGVPGVPGAIPGIGGIAGVGTPAAAAA
AAAAAKAAKYGAAAGLVPGGPGFGPGVVGVPGAGV
PGVGVPGAGIPVVPGAGIPGAAVPGVVSPEAAAKA
AAKAAKYGARPGVGVGGIPTYGVGAGGFPGFGVGV
GGIPGVAGVPSVGGVPGVGGVPGVGISPEAQAAAA
AKAAKYGVGTPAAAAAKAAAKAAQFGLVPGVGVAP
GVGVAPGVGVAPGVGLAPGVGVAPGVGVAPGVGVA
PGIGPGGVAAAAKSAAKVAAKAQLRAAAGLGAGIP
GLGVGVGVPGLGVGAGVPGLGVGAGVPGFGAVPGA
LAAAKAAKYGAAVPGVLGGLGALGGVGIPGGVVGA
GPAAAAAAAKAAAKAAQFGLVGAAGLGGLGVGGLG
VPGVGGLGGIPPAAAAKAAKYGAAGLGGVLGGAGQ
FPLGGVAARPGFGLSPIFPGGACLGKACGRKRK;
(SEQ ID NO: 38)
MGGVPGAVPGGVPGGVFYPGAGFGAVPGGVADAAA
AYKAAKAGAGLGGVPGVGGLGVSAGAVVPQPGAGV
KPGKVPGVGLPGVYPGFGAVPGARFPGVGVLPGVP
TGAGVKPKAPGVGGAFAGIPGVGPFGGPQPGVPLG
YPIKAPKLPGGYGLPYTTGKLPYGYGPGGVAAAGK
AGYPTGTGVGPQAAAAAAAKAAAKFGAGAAGFGAV
PGVGGAGVPGVPGAIPGIGGIAGVGTPAAAAAAAA
AAKAAKYGAAAGLVPGGPGFGPGVVGVPGFGAVPG
VGVPGAGIPVVPGAGIPGAAGFGAVSPEAAAKAAA
KAAKYGARPGVGVGGIPTYGVGAGFFPGFGVGVGG
IPGVAGVPSVGGVPGVGGVPGVGISPEAQAAAAAK
AAKYGVGTPAAAAAKAAAKAAQFGLVPGVGVAPGV
GVAPGVGVAPGVGLAPGVGVAPGVGVAPGVGVAPG
IGPGGVAAAAKSAAKVAAKAQLRAAAGLGAGIPGL
GVGVGVPGLGVGAGVPGLGVGAGVPGFGAVPGALA
AAKAAKYGAVPGVLGGLGALGGVGIPGGVVGAGPA
AAAAAAKAAAKAAQFGLVGAAGLGGLGVGGLGVPG
VGGLGGIPPAAAAKAAKYGAAGLGGVLGGAGQFPL
GGVAARPGFGLSPIFPGGACLGKACGRKRK;
(SEQ ID NO: 39)
SAMGGVPGALAAAKAAKYGAAVPGVLGGLGALGGV
GIPGGVVGAGPAAAAAAAKAAAKAAQFGLVGAAGL
GGLGVGGLGVPGVGGLGGIPPAAAAKAAKYGAAGL
GGVLGGAGQFPLGGVAARPGFGLSPIFPGGACLGK
ACGRKRK;
(SEQ ID NO: 40)
SAMGALVGLGVPGLGVGAGVPGFGAGADEGVRRSL
SPELREGDPSSSQHLPSTPSSPRVPGALAAAKAAK
YGAAVPGVLGGLGALGGVGIPGGVVGAGPAAAAAA
AKAAAKAAQFGLVGAAGLGGLGVGGLGVPGVGGLG
GIPPAAAAKAAKYGAAGLGGVLGGAGQFPLGGVAA
RPGFGLSPIFPGGACLGKACGRKRK;
(SEQ ID NO: 41)
GIPPAAAAKAAKYGAAGLGGVLGGAGQFPLGGVAA
RPGFGLSPIFPGGACLGKACGRKRK;
(SEQ ID NO: 42)
GAAGLGGVLGGAGQFPLGGVAARPGFGLSPIFPGG
ACLGKACGRKRK;
(SEQ ID NO: 43)
GADEGVRRSLSPELREGDPSSSQHLPSTPSSPRV;
(SEQ ID NO: 44)
GADEGVRRSLSPELREGDPSSSQHLPSTPSSPRF;
(SEQ ID NO: 45)
AAAGLGAGIPGLGVGVGVPGLGVGAGVPGLGVGAG
VPGFGAGADEGVRRSLSPELREGDPSSSQHLPSTP
SSPRVPGALAAAKAAKYGAAVPGVLGGLGALGGVG
IPGGVVGAGPAAAAAAAKAAAKAAQFGLVGAAGLG
GLGVGGLGVPGVGGLGGIPPAAAAKAAKYGAAGLG
GVLGGAGQFPLGGVAARPGFGLSPIFPGGACLGKA
CGRKRK;
and
(SEQ ID NO: 46)
AAAGLGAGIPGLGVGVGVPGLGVGAGVPGLGVGAG
VPGFGAVPGALAAAKAAKYGAAVPGVLGGLGALGG
VGIPGGVVGAGPAAAAAAAKAAAKAAQFGLVGAAG
LGGLGVGGLGVPGVGGLGGIPPAAAAKAAKYGAAG
LGGVLGGAGQFPLGGVAARPGFGLSPIFPGGACLG
KACGRKRK.

### Hybrid Polymeric Material

The hybrid polymeric material comprises copolymers of tropoelastin and a polyol-polycarboxylic acid copolymer. That is, the tropoelastin and polyol-polycarboxylic acid copolymer are linked in a manner where the tropoelastin and polyol-polycarboxylic acid form a stable material that may not substantially leach tropoelastin or polyol-polycarboxylic acid copolymer from the hybrid polymeric material when placed in PBS at pH 7 at standard temperature and pressure for one hour (i.e. the hybrid polymeric material may not lose more than about 50%, 40%, 30%, 20%, or 10% of its dry weight when placed in PBS at pH 7 and standard temperature and pressure for 1 hour). The hybrid polymeric material may be a solid material at standard pressure and temperature. The skilled person will understand that the hybrid polymeric material may be any size or shape, and it may have any structure, microstructure, or morphology depending on its intended application. It may, for example, have a sheet or tubular structure. It may, for example, comprise fibers. It may have a porous structure. In certain embodiments it may have a non-porous structure.

The hybrid polymeric material may comprise a copolymer of tropoelastin and a polymer selected from the group consisting of poly(glycerol succinate), poly(glycerol glutarate), poly(glycerol adipate), poly(glycerol pimelate), poly(glycerol suberate), polyglycerol (azelate), poly(glycerol sebacate), poly(glycerol undecanoate), poly(glycerol dodecanoate), poly(citrate glyceride), poly(xylitol sebacate), poly(pentraerythritol sebacate), and combinations thereof. It may, for example, comprise a copolymer of tropoelastin and poly(glycerol sebacate).

The mass ratio of the tropoelastin to the polyol-polycarboxylic acid copolymer may be from about 1:99 to about 99:1, or it may be from about 10:90 to about 99:1, about 20:80 to about 99:1, about 30:70 to about 99:1, about 40:60 to about 99:1, about 50:50 to about 99:1, about 1:99 to about 90:10, about 1:99 to about 80:20, about 1:99 to about 70:30, about 10:90 to about 90:10, about 20:80 to about 80:20, about 30:70 to about 80:20, about 40:60 to about 80:20, about 50:50 to about 80:20, about 50:50 to about 70:30, or about 50:50 to about 90:10.

The hybrid polymeric material may additionally comprise other extracellular matrix proteins (i.e. other than the tropoelastin) or derivatives thereof, pharmaceutically acceptable excipients, salts, and/or one or more therapeutic agents. The other extracellular matrix proteins may, for example, be selected from the group consisting of collagen, gelatin, and combinations thereof. The therapeutic agents may, for example, assist in tissue regeneration processes. Suitable agents may be selected from, for example, cells, anticoagulants, growth factors, cytokines, enzymes, hormones, extracellular matrix materials, vitamins, other small molecules that promote or assist in tissue regeneration, and combinations thereof. Additional agent(s) may be added before, during or after heat treatment. The skilled person will understand that the decision on when to add the agent(s) may be in part determined by resistance of the respective agent to damage by heat. For example, cells may be added after heat treatment.

In the case where the hybrid polymeric material comprises fibers, the fibers may have an average fiber width of from about 5 nm to about 10 μm, or from about 5 nm to about 5 μm, about 5 nm to about 2000 nm, about 5 nm to about 1500 nm, about 5 nm to about 1000 nm, about 5 nm to about 900 nm, about 5 nm to about 800 nm, about 5 nm to about 700 nm, about 5 nm to about 600 nm, about 20 nm to about 10 μm, about 50 nm to about 10 μm, about 100 nm to about 10 μm, about 200 nm to about 10 μm, about 100 nm to about 1000 nm, about 200 nm to about 800 nm, about 200 nm to about 600 nm, about 200 nm to about 500 nm, about 200 nm to about 400 nm, or about 200 nm to about 600 nm. It may have an average fiber width of, for example, about 5 nm, 10 nm, 20 nm, 50 nm, 100 nm, 200 nm, 300 nm, 400 nm, 500 nm, 600 nm, 800 nm, 1000 nm, 1500 nm, 2000 nm, 5000 nm, or 10000 nm.

In the case where the hybrid polymeric material has a porous structure, the hybrid polymeric material may have an average pore size of from about 0.05 μm to about 1000 μm, or from about 0.05 μm to about 500 μm, about 0.05 μm to about 200 μm, about 0.05 μm to about 100 μm, about 0.05 μm to about 50 μm, about 0.05 μm to about 20 μm, about 0.05 μm to about 10 μm, or from about 0.05 μm to about 5 μm, about 0.05 μm to about 4 μm, about 0.05 μm to about 3 μm, about 0.05 μm to about 2 μm, about 0.1 μm to about 100 μm, about 0.2 μm to about 100 μm, about 0.5 μm to about 100 μm, about 0.75 μm to about 100 μm, about 1 μm to about 100 μm, about 2 μm to about 100 μm, about 5 μm to about 100 μm, about 7.5 μm to about 100 μm, about 0.1 μm to about 10 μm, about 0.2 μm to about 10 μm, about 0.5 μm to about 10 μm, about 0.75 μm to about 10 μm, about 0.2 μm to about 2 μm, about 0.4 μm to about 2 μm, about 0.6 μm to about 2 μm, about 0.8 μm to about 2 μm, about 0.2 μm to about 1.5 μm, about 0.2 μm to about 1.4 μm, about 0.2 μm to about 1.2 μm, about 0.4 μm to about 1.2 μm, about 0.6 μm to about 1.2 μm, about 0.7 μm to about 1.2 μm, or about 0.6 μm to about 1.5 μm. It may have an average pore size of about 0.05 m, 0.1 μm, 0.2 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 3 μm, 4 μm, 5 μm, 6 μm, 8 μm, 10 μm, 11 μm, 12 μm, 15 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm.

In the case where the hybrid polymeric material has a porous structure, the hybrid polymeric material may have a percentage porosity of from about 0.5% to about 95%, or from about 0.5% to about 90%, about 0.5% to about 80%, about 0.5% to about 70%, about 0.5% to about 60%, about 0.5% to about 50%, about 1% to about 95%, about 5% to about 95%, about 10% to about 95%, about 20% to about 95%, about 30% to about 95%, about 40% to about 95%, about 20% to about 80%, about 30% to about 80%, about 20% to about 70%, or about 30% to about 60%. It may have a percentage porosity of about 0.5%, 1%, 2%, 5%, 10%, 20%, 30%, 35%, 40%, 45%, 50%, 55%, 60%, 65%, 70%, 80%, 90%, or 95%.

The hybrid polymeric material may have a Young's modulus of from about 0.01 MPa to about 100 MPa, or it may be from about 0.01 MPa to about 80 MPa, about 0.01 MPa to about 50 MPa, about 0.01 MPa to about 40 MPa, about 0.01 MPa to about 30 MPa, about 0.1 MPa to about 80 MPa, about 0.1 MPa to about 50 MPa, about 0.1 MPa to about 40 MPa, about 0.1 MPa to about 30 MPa, about 0.5 MPa to about 100 MPa, about 1 MPa to about 100 MPa, about 1 MPa to about 50 MPa, about 1 MPa to about 40 MPa, or about 1 MPa to about 30 MPa. It may be, for example, about 0.01 MPa, 0.02 MPa, 0.05 MPa, 0.1 MPa, 0.2 MPa, 0.5 MPa, 1 MPa, 1.1 MPa, 1.2 MPa, 1.5 MPa, 2 MPa, 5 MPa, 10 MPa, 11 MPa, 12 MPa, 15 MPa, 20 MPa, 21 MPa, 22 MPa, 25 MPa, 30 MPa, 35 MPa, 40 MPa, 50 MPa, 60 MPa, 70 MPa, 80 MPa, 90 MPa, or 100 MPa.

The hybrid polymeric material may have an ultimate tensile strength of from about 0.01 MPa to about 100 MPa, or it may be from about 0.01 MPa to about 80 MPa, about 0.01 MPa to about 50 MPa, about 0.01 MPa to about 40 MPa, about 0.01 MPa to about 30 MPa, about 0.1 MPa to about 80 MPa, about 0.1 MPa to about 50 MPa, about 0.1 MPa to about 40 MPa, about 0.1 MPa to about 30 MPa, about 0.5 MPa to about 100 MPa, about 1 MPa to about 100 MPa, about 1 MPa to about 50 MPa, about 1 MPa to about 40 MPa, about 1 MPa to about 30 MPa, about 1 MPa to about 20 MPa, or about 2 MPa to about 10 MPa. It may be, for example, about 0.01 MPa, 0.02 MPa, 0.05 MPa, 0.1 MPa, 0.2 MPa, 0.5 MPa, 1 MPa, 1.1 MPa, 1.2 MPa, 1.5 MPa, 2 MPa, 3 MPa, 4 MPa, 5 MPa, 6 MPa, 7 MPa, 8 MPa, 9 MPa, 10 MPa, 11 MPa, 12 MPa, 15 MPa, 20 MPa, 21 MPa, 22 MPa, 25 MPa, 30 MPa, 35 MPa, 40 MPa, 50 MPa, 60 MPa, 70 MPa, 80 MPa, 90 MPa, or 100 MPa.

The hybrid polymeric material may have a percentage elongation at failure of from about 30% to about 300%, or from about 40% to about 300%, about 30% to about 200%, about 30% to about 150%, about 40% to about 150%, or about 40% to about 110%. It may be, for example, about 30%, 35%, 40%, 45%, 50%, 55%, 60%, 70%, 75%, 80%, 90%, 100%, 110%, 120%, 150%, 200%, 250%, or 300%.

The hybrid polymeric material may be stable when incubated at 37° C. in PBS. It may lose less than about 50% of its mass when incubated at 37° C. in PBS at pH 7 for 1 week, or less than 40%, 35%, 30%, 25%, 20%, 15%, 10%, 9%, 8%, or 7% of its mass when incubated at 37° C. in PBS at pH 7 for 1 week.

The hybrid polymeric material may swell when placed in a liquid. It may swell when placed in water, or an aqueous solution, such as PBS. It may form a hydrogel when placed in water, or an aqueous solution. It may swell to from about 101% to about 500% of its dry mass when placed in PBS, or from about 101% to about 400%, about 101% to about 300%, about 101% to about 200%, about 101% to about 190%, about 101% to about 180%, or about 110% to about 170% of its dry mass when placed in PBS. It may, for example, swell to from about 101%, 102%, 105%, 110%, 120%, 130%, 140%, 150%, 160%, 170%, 180%, 190%, 200%, 210%, 220%, 250%, 300%, 350%, 400%, 450%, or 500% of its dry mass when placed in PBS.

The hybrid polymeric material may be used for a variety of articles. For example, it may be used for an implant, such as a tissue scaffold. It may be used in a component of an implant. It may be used in a component of a tissue scaffold. It may be used, for example, for a vascular graft, a heart valve, nerve guide, surgical patch, or a wound-healing scaffold.

### Tissue Scaffold

Disclosed herein is a tissue scaffold comprising the hybrid polymeric material as hereinbefore described. The tissue scaffold may be, for example, a vascular graft, a heart valve, nerve guide, surgical patch, or a wound-healing scaffold. The skilled person will understand that the size or shape of the tissue scaffold will depend upon its intended purpose. For example, a vascular graft may have a tubular shape and have a similar size to the vascular component (e.g. artery, vein etc) which the graft is intended to replace. In contrast, a wound healing scaffold may have a planar shape, with its size dependent upon the wound size intended to be treated with the scaffold. The tissue scaffold may comprise fibers. It may have a porous structure. In certain embodiments it may have a non-porous structure.

The tissue scaffold may have a Young's modulus of from about 0.01 MPa to about 100 MPa, or it may be from about 0.01 MPa to about 80 MPa, about 0.01 MPa to about 50 MPa, about 0.01 MPa to about 40 MPa, about 0.01 MPa to about 30 MPa, about 0.1 MPa to about 80 MPa, about 0.1 MPa to about 50 MPa, about 0.1 MPa to about 40 MPa, about 0.1 MPa to about 30 MPa, about 0.5 MPa to about 100 MPa, about 1 MPa to about 100 MPa, about 1 MPa to about 50 MPa, about 1 MPa to about 40 MPa, or about 1 MPa to about 30 MPa. It may be, for example, about 0.01 MPa, 0.01 MPa, 0.02 MPa, 0.03 MPa, 0.04 MPa, 0.05 MPa, 0.06 MPa, 0.07 MPa, 0.08 MPa, 0.09 MPa, 0.1 MPa, 0.2 MPa, 0.3 MPa, 0.4 MPa, 0.5 MPa, 0.6 MPa, 0.7 MPa, 0.8 MPa, 0.9 MPa, 1.0 MPa, 1.1 MPa, 1.2 MPa, 1.5 MPa, 2.0 MPa, 3.0 MPa, 4.0 MPa, 5.0 MPa, 6.0 MPa, 7.0 MPa, 8.0 MPa, 9.0 MPa, 10 MPa, 11.0 MPa, 12.0 MPa, 15.0 MPa, 20 MPa, 21 MPa, 22 MPa, 25 MPa, 30 MPa, 35 MPa, 40 MPa, 50 MPa, 60 MPa, 70 MPa, 80 MPa, 90 MPa, or 100 MPa.

The tissue scaffold may have an ultimate tensile strength of from about 0.01 MPa to about 100 MPa, or it may be from about 0.01 MPa to about 80 MPa, about 0.01 MPa to about 50 MPa, about 0.01 MPa to about 40 MPa, about 0.01 MPa to about 30 MPa, about 0.1 MPa to about 80 MPa, about 0.1 MPa to about 50 MPa, about 0.1 MPa to about 40 MPa, about 0.1 MPa to about 30 MPa, about 0.5 MPa to about 100 MPa, about 1 MPa to about 100 MPa, about 1 MPa to about 50 MPa, about 1 MPa to about 40 MPa, about 1 MPa to about 30 MPa, about 1 MPa to about 20 MPa, or about 2 MPa to about 10 MPa. It may be, for example, about 0.01 MPa, 0.01 MPa, 0.02 MPa, 0.03 MPa, 0.04 MPa, 0.05 MPa, 0.06 MPa, 0.07 MPa, 0.08 MPa, 0.09 MPa, 0.1 MPa, 0.2 MPa, 0.3 MPa, 0.4 MPa, 0.5 MPa, 0.6 MPa, 0.7 MPa, 0.8 MPa, 0.9 MPa, 1.0 MPa, 1.1 MPa, 1.2 MPa, 1.5 MPa, 2.0 MPa, 3.0 MPa, 4.0 MPa, 5.0 MPa, 6.0 MPa, 7.0 MPa, 8.0 MPa, 9.0 MPa, 10 MPa, 11.0 MPa, 12.0 MPa, 15.0 MPa, 20 MPa, 21 MPa, 22 MPa, 25 MPa, 30 MPa, 35 MPa, 40 MPa, 50 MPa, 60 MPa, 70 MPa, 80 MPa, 90 MPa, or 100 MPa.

The tissue scaffold may have a percentage elongation at failure of from about 30% to about 300%, or from about 40% to about 300%, about 30% to about 200%, about 30% to about 150%, about 40% to about 150%, or about 40% to about 110%. It may be, for example, about 30%, 35%, 40%, 45%, 50%, 55%, 60%, 70%, 75%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, or 400%.

The tissue scaffold may be stable when incubated at 37° C. in PBS. It may lose less than about 40% of its mass when incubated at 37° C. in PBS for 1 week, or less than 40%, 35%, 30%, 25%, 20%, 15%, 10%, 9%, 8%, or 7% of its mass when incubated at 37° C. in PBS for 1 week.

In the case where the tissue scaffold comprises fibers, the fibers may have an average fiber width of from about 5 nm to about 10 μm, or from about 5 nm to about 5000 nm, 5 nm to about 2000 nm, about 5 nm to about 1500 nm, about 5 nm to about 1000 nm, about 5 nm to about 900 nm, about 5 nm to about 800 nm, about 5 nm to about 700 nm, about 5 nm to about 600 nm, about 20 nm to about 10 μm, about 50 nm to about 10 μm, about 100 nm to about 10 μm, about 200 nm to about 10 μm, about 100 nm to about 1000 nm, about 200 nm to about 800 nm, about 200 nm to about 600 nm, about 200 nm to about 500 nm, about 200 nm to about 400 nm, or about 200 nm to about 600 nm. It may have an average fiber width of, for example, about 5 nm, 10 nm, 20 nm, 30 nm, 40 nm, 50 nm, 60 nm, 70 nm, 80 nm, 90 nm, 100 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 700 nm, 800 nm, 900 nm, 1 μm, 2 μm, 3 μm, 4 μm, 5 μm, 6 μm, 7 μm, 8 μm, 9 μm, or 10 μm.

In the case where the tissue scaffold has a porous structure, the tissue scaffold may have an average pore size of from about 0.05 μm to about 1000 μm, or from about 0.05 μm to about 500 μm, about 0.05 μm to about 200 μm, about 0.05 μm to about 100 μm, about 0.05 μm to about 50 μm, about 0.05 μm to about 20 μm, about 0.05 μm to about 10 μm, or from about 0.05 μm to about 5 μm, about 0.05 μm to about 4 μm, about 0.05 μm to about 3 μm, about 0.05 μm to about 2 μm, about 0.1 μm to about 100 μm, about 0.2 μm to about 100 μm, about 0.5 μm to about 100 μm, about 0.75 μm to about 100 μm, about 1 μm to about 100 μm, about 2 μm to about 100 μm, about 5 μm to about 100 μm, about 7.5 μm to about 100 μm, about 0.1 μm to about 10 μm, about 0.2 μm to about 10 μm, about 0.5 μm to about 10 μm, about 0.75 μm to about 10 μm, about 0.2 μm to about 2 μm, about 0.4 μm to about 2 μm, about 0.6 μm to about 2 μm, about 0.8 μm to about 2 μm, about 0.2 μm to about 1.5 μm, about 0.2 μm to about 1.4 μm, about 0.2 μm to about 1.2 μm, about 0.4 μm to about 1.2 μm, about 0.6 μm to about 1.2 μm, about 0.7 μm to about 1.2 μm, or about 0.6 μm to about 1.5 μm. It may have an average pore size of about 50 nm, 60 nm, 70 nm, 80 nm, 90 nm, 100 nm, 200 nm, 300 nm, 400 nm, 500 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, 1 μm, 1.1 μm, 1.2 μm, 1.3 μm, 1.4 μm, 1.5 μm, 1.6 μm, 1.7 μm, 1.8 μm, 1.9 μm, 2 μm, 3 μm, 4 μm, 5 μm, 6 μm, 7 μm, 8 μm, 9 μm, or 10 μm.

In the case where the tissue scaffold has a porous structure, the tissue scaffold may have a percentage porosity of from about 0.5% to about 95%, or from about 0.5% to about 90%, about 0.5% to about 80%, about 0.5% to about 70%, about 0.5% to about 60%, about 0.5% to about 50%, about 1% to about 95%, about 5% to about 95%, about 10% to about 95%, about 20% to about 95%, about 30% to about 95%, about 40% to about 95%, about 20% to about 80%, about 30% to about 80%, about 20% to about 70%, or about 30% to about 60%. It may have a percentage porosity of about 0.5%, 1%, 2%, 5%, 10%, 20%, 30%, 35%, 40%, 45%, 50%, 55%, 60%, 65%, 70%, 80%, 90%, or 95%.

The tissue scaffold may swell when placed in a liquid. It may swell when placed in water, or an aqueous solution, such as PBS. It may form a hydrogel when placed in water, or an aqueous solution. It may swell to from about 101% to about 500% of its dry mass when placed in PBS, or from about 101% to about 400%, about 101% to about 300%, about 101% to about 200%, about 101% to about 190%, about 101% to about 180%, or about 110% to about 170% of its dry mass when placed in PBS. It may, for example, swell to from about 101%, 102%, 105%, 110%, 120%, 130%, 140%, 150%, 160%, 170%, 180%, 190%, 200%, 210%, 220%, 250%, 300%, 350%, 400%, 450%, or 500% of its dry mass when placed in PBS.

### Method for Producing a Hybrid Polymeric Material

Disclosed herein is a method for producing a hybrid polymeric material. The method comprises the steps of: (A) providing a mixture comprising a tropoelastin and a copolymer of a polyol monomer and a polycarboxylic acid monomer, and (B) heating the mixture to form the hybrid polymeric material. The tropoelastin, polyol monomer, and polycarboxylic acid monomer are as hereinbefore described.

The heating may be at a temperature of from about 50° C. to about 220° C., or from about 60° C. to about 220° C., about 70° C. to about 220° C., about 80° C. to about 220° C., about 90° C. to about 220° C., about 100° C. to about 220° C., about 110° C. to about 220° C., about 120° C. to about 220° C., about 130° C. to about 220° C., about 140° C. to about 220° C., about 150° C. to about 220° C., about 100° C. to about 200° C., about 120° C. to about 200° C., about 140° C. to about 200° C., or about 140° C. to about 180° C. It may be, for example, at about 50° C., 60° C., 70° C., 80° C., 90° C., 100° C., 110° C., 120° C., 130° C., 140° C., 150° C., 160° C., 170° C., 180° C., 190° C., 200° C., 210° C., or 220° C.

The heating may be for a period of greater than about 10 minutes, about 20 minutes, about 30 minutes, 40 minutes, 50 minutes, 1 hour, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours. It may be for a period of from about 10 minutes to about 24 hours, or from about 10 minutes to about 20 hours, about 10 minutes to about 18 hours, about 10 minutes to about 16 hours, about 20 minutes to about 20 hours, about 30 minutes to about 20 hours, about 40 minutes to about 20 hours, about 50 minutes to about 20 hours, about 60 minutes to about 20 hours, about 1.5 hours to about 20 hours, about 2 hours to about 20 hours, about 4 hours to about 20 hours, about 4 hours to about 20 hours, about 8 hours to about 20 hours, about 10 hours to about 20 hours, about 12 hours to about 20 hours, about 14 hours to about 20 hours, about 12 hours to about 18 hours, or about 14 hours to about 18 hours. It may be for a period of, for example, about 10 minutes, 20 minutes, 30 minutes, 40 minutes, 50 minutes, 1 hour, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.

Advantageously, the hybrid polymeric material may be cured at atmospheric pressure. Accordingly, the method may be performed substantially at atmospheric pressure. It may be performed, for example, at a pressure of about 1 atmosphere.

The mixture may comprise a solvent, and the method may further comprise a step of removing (e.g., substantially removing) or reducing the amount of solvent prior to step (B). The solvent may be an organic solvent. It may be an aqueous solvent. It may be a polar organic solvent. It may be a polar organic solvent having a boiling point below 80° C. It may be an alcohol. It may be a halogenated alcohol. It may be selected from the group consisting of hexafluoro-2-propanol, tetrahydrofuran, trifluoroacetic acid, N,N-dimethylformamide, and combinations thereof. It may be, for example, hexafluoro-2-propanol.

The mixture may further comprise other extracellular matrix proteins (i.e. other than the tropoelastin) or derivatives thereof, pharmaceutically acceptable excipients, salts, and/or one or more therapeutic agents. The other extracellular matrix proteins may, for example, be selected from the group consisting of collagen, gelatin, and combinations thereof. The therapeutic agents may, for example, assist in tissue regeneration processes. Suitable agents may be selected from, for example, cells, anticoagulants, growth factors, cytokines, enzymes, hormones, extracellular matrix materials, vitamins, other small molecules that promote or assist in tissue regeneration, and combinations thereof.

The method may comprise a step of adding other extracellular matrix proteins (i.e. other than the tropoelastin) or derivatives thereof, pharmaceutically acceptable excipients, salts, and/or one or more therapeutic agents to the mixture prior to or after the heating step.

The mass ratio of the tropoelastin to the polyol-polycarboxylic acid copolymer in the mixture may be from about 1:99 to about 99:1, or it may be from about 10:90 to about 99:1, about 20:80 to about 99:1, about 30:70 to about 99:1, about 40:60 to about 99:1, about 50:50 to about 99:1, about 1:99 to about 90:10, about 1:99 to about 80:20, about 1:99 to about 70:30, about 10:90 to about 90:10, about 20:80 to about 80:20, about 30:70 to about 80:20, about 40:60 to about 80:20, about 50:50 to about 80:20, about 50:50 to about 70:30, or about 50:50 to about 90:10.

The weight percentage of the tropoelastin in the mixture may be from about 1 wt % to about 99 wt %, or it may be from about 1 wt % to about 95 wt %, about 1 wt % to about 90 wt %, about 1 wt % to about 80 wt %, about 1 wt % to about 70 wt %, about 1 wt % to about 60 wt %, about 1 wt % to about 50 wt %, about 1 wt % to about 40 wt %, about 1 wt % to about 30 wt %, about 1 wt % to about 20 wt %, about 1 wt % to about 10 wt %, about 5 wt % to about 20 wt %, or about 5 wt % to about 15 wt %. It may be, for example, about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 20 wt %, 25 wt %, 30 wt %, 40 wt %, 50 wt %, 60 wt %, 70 wt %, 80 wt %, 90 wt %, 95 wt %, or 99 wt %.

The weight percentage of the polyol-polycarboxylic acid copolymer in the mixture may be from about 1 wt % to about 99 wt %, or it may be from about 1 wt % to about 95 wt %, about 1 wt % to about 90 wt %, about 1 wt % to about 80 wt %, about 1 wt % to about 70 wt %, about 1 wt % to about 60 wt %, about 1 wt % to about 50 wt %, about 1 wt % to about 40 wt %, about 1 wt % to about 30 wt %, about 1 wt % to about 20 wt %, about 1 wt % to about 10 wt %, about 5 wt % to about 20 wt %, or about 5 wt % to about 15 wt %. It may be, for example, about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 20 wt %, 25 wt %, 30 wt %, 40 wt %, 50 wt %, 60 wt %, 70 wt %, 80 wt %, 90 wt %, 95 wt %, or 99 wt %.

The boiling point of the solvent may be below 120° C., or it may be below 110° C., 100° C., 90° C., 80° C., 70° C., or 60° C. It may be from about 10° C. to about 120° C., or from about 10° C. to about 100° C., about 10° C. to about 80° C., about 20° C. to about 120° C., about 40° C. to about 120° C., about 50° C. to about 80° C., or about 50° C. to about 70° C. It may be, for example, about 10° C., 20° C., 30° C., 40° C., 50° C., 60° C., 70° C., 80° C., 90° C., 100° C., 110° C., or 120° C.

The solvent may have a vapour pressure of more than about 5 kPa at 20° C., or more than about 7 kPa, 8 kPa, 9 kPa, 10 kPa, 11 kPa, 12 kPa, 13 kPa, 14 kPa, or 15 kPa at 20° C. It may have a vapour pressure of from about 5 kPa to about 50 kPa at 20° C., or it may be from about 5 kPa to about 30 kPa, about 5 kPa to about 20 kPa, about 10 kPa to about 50 kPa, about 15 kPa to about 50 kPa, or about 10 kPa to about 20 kPa at 20° C. It may be, for example, about 5, 6, 7, 8, 9, 10, 11, 12, 13, 14, 15, 16, 17, 18, 19, 20, 21, 22, 25, 30, 35, 40, 45, or 50 kPa at 20° C.

In certain embodiments, the method comprises a step of depositing, e.g., casting the mixture as a film. In such embodiments, solvent, if present in the mixture, is preferably removed (e.g. substantially removed) prior to step (B). The depositing may be through spin coating, spray coating, dip coating, drop casting, roller coating, printing or any other suitable deposition method.

In the case where the depositing is through spin coating, the spin coating may be for more than about 10 seconds, or it may be for more than about 15 seconds, 20 seconds, 30 seconds, 40 seconds, 50 seconds, 60 seconds, 90 seconds, 120 seconds, 150 seconds, 150 seconds, or 180 seconds. It may be from about 10 seconds to about 300 seconds, or from about 30 seconds to about 300 seconds, about 60 seconds to about 300 seconds, about 120 seconds to about 300 seconds, about 240 seconds to about 300 seconds, about 10 seconds to about 30 seconds, about 30 seconds to about 60 seconds, about 60 seconds to about 120 seconds, about 120 seconds to about 240 seconds, about 10 seconds to about 20 seconds, about 10 seconds to about 40 seconds, about 10 seconds to about 60 seconds, about 10 seconds to about 120 seconds, about 10 seconds to about 240 seconds, about 30 seconds to about 90 seconds, about 90 seconds to about 240 seconds, about 60 seconds to about 120 seconds, or about 60 seconds to about 240 seconds. It may be for example about 10 seconds, 15 seconds, 20 seconds, 25 seconds, 30 seconds, 40 seconds, 50 seconds, 60 seconds, 90 seconds, 120 seconds, 180 seconds, 240 seconds, or 300 seconds. The spin coating may be performed at above about 100 rpm, or above about 200 rpm, 300 rpm, 500 rpm, 700 rpm, 1000 rpm, 1500 rpm, 2000 rpm, 3000 rpm, 4000 rpm, or 5000 rpm. It may be performed from about 10 rpm to about 10000 rpm, or from about 50 rpm to about 10000, about 100 rpm to about 10000 rpm, about 250 rpm to about 10000 rpm, about 500 rpm to about 10000 rpm, about 1000 rpm to about 10000 rpm, about 2000 rpm to about 10000 rpm, about 5000 rpm to about 10000 rpm, about 10 rpm to about 100 rpm, about 100 rpm to about 500 rpm, about 500 rpm to about 1000 rpm, about 1000 rpm to about 2000 rpm, about 2000 rpm to about 3000 rpm, about 3000 rpm to about 4000 rpm, about 4000 rpm to about 5000 rpm, about 5000 rpm to about 7500 rpm, about 10 rpm to about 50 rpm, about 10 rpm to about 200 rpm, about 10 rpm to about 400 rpm, about 10 rpm to about 500 rpm, about 10 rpm to about 750 rpm, about 10 rpm to about 1000 rpm, about 10 rpm to about 2000 rpm, about 10 to about 3000 rpm, about 10 rpm to about 5000 rpm, about 10 rpm to about 7500 rpm, about 200 rpm to about 5000 rpm, about 500 rpm to about 5000 rpm, about 1000 rpm to about 5000 rpm, or about 2000 rpm to about 4000 rpm. It may for example be performed at about 10 rpm, 20 rpm, 50 rpm, 100 rpm, 200 rpm, 500 rpm, 700 rpm, 1000 rpm, 1500 rpm, 2000 rpm, 2500 rpm, 3000 rpm, 3500 rpm, 4000 rpm, 4500 rpm, 5000 rpm, 6000 rpm, 7000 rpm, 8000 rpm, 9000 rpm, or 10000 rpm. The spin coating may be performed at below 100% relative humidity, or at below 90%, 80%, 70%, 60%, 50%, 40%, 30%, 20%, or 10% relative humidity. It may be performed from about 0% to about 100% relative humidity, or from about 0% to about 90%, about 0 to about 80%, about 0 to about 60%, about 0 to about 40%, about 0 to about 20%, about 80 to about 100%, about 60 to about 100%, about 40 to about 100%, about 20 to about 100%, or about 20 to about 60% relative humidity. It may be performed at about 0%, 10%, 20%, 30%, 40%, 50%, 60%, 70%, 80%, 90%, or 100% relative humidity. The skilled person will be able to select suitable spin coating conditions to afford desirable properties for the deposited film. For example, the speed and time may depend on the desired film thickness.

In the case where the depositing is through dip coating, the dip coating may be performed with a withdrawal velocity of greater than about 0.5 mm/s, or greater than about 1 mm/s, 2 mm/s, 5 mm/s, 10 mm/s, 20 mm/s, or 50 mm/s. It may be from about 0.5 to about 100 mm/s, or from about 0.5 to about 1 mm/s, about 0.5 to about 2 mm/s, about 0.5 to about 5 mm/s, about 0.5 to about 10 mm/s, about 0.5 to about 20 mm/s, about 0.5 to about 50 mm/s, about 50 to about 100 mm/s, about 20 to about 100 mm/s, about 10 to about 100 mm/s, about 5 to about 100 mm/s, about 2 to about 100 mm/s, about 1 to about 100 mm/s, about 1 to about 2 mm/s, about 2 to about 5 mm/s, about 5 to about 10 mm/s, about 10 to about 20 mm/s, or about 20 to about 50 mm/s. It may be for example about 0.5 mm/s, 1 mm/s, 2 mm/s, 5 mm/s, 10 mm/s, 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, or 100 mm/s. The dip coating may be performed at below 100% relative humidity, or at below 90%, 80%, 70%, 60%, 50%, 40%, 30%, 20%, or 10% relative humidity. It may be performed from about 0% to about 100% relative humidity, or from about 0% to about 90%, about 0% to about 80%, about 0% to about 60%, about 0% to about 40%, about 0% to about 20%, about 80% to about 100%, about 60% to about 100%, about 40% to about 100%, about 20% to about 100%, or about 20% to about 60% relative humidity. It may be performed at about 0%, 10%, 20%, 30%, 40%, 50%, 60%, 70%, 80%, 90%, or 100% relative humidity. The skilled person will be able to select suitable dip coating conditions to afford desirable properties for the deposited film. For example, the withdrawal velocity may depend on the desired film thickness.

In the case where the depositing is through spray coating, the spray coating may be performed with a dispensing flow rate of greater than about 0.5 μL/s, or greater than about 1, 2, 5, 10, 20, 50, 100, 200 or 500 μL/s. It may be from about 0.5 μL/s to about 1000 μL/s, or from about 0.5 μL/s to about 1 μL/s, about 0.5 μL/s to about 2 μL/s, about 0.5 μL/s to about 5 μL/s, about 0.5 μL/s to about 10 μL/s, about 0.5 μL/s to about 20 μL/s, about 0.5 μL/s to about 50 μL/s, about 0.5 μL/s to about 100 μL/s, about 0.5 μL/s to about 200 μL/s, about 0.5 μL/s to about 500 μL/s, about 500 μL/s to about 1000 μL/s, about 200 μL/s to about 1000 μL/s, about 100 μL/s to about 1000 μL/s, about 50 μL/s to about 1000 μL/s, about 20 μL/s to about 1000 μL/s, about 10 μL/s to about 1000 μL/s, about 5 μL/s to about 1000 μL/s, about 2 μL/s to about 1000 μL/s, about 1 μL/s to about 1000 μL/s, about 1 μL/s to about 2 μL/s, about 2 μL/s to about 5 μL/s, about 5 μL/s to about 10 μL/s, about 10 μL/s to about 20 μL/s, about 20 μL/s to about 50 μL/s, about 50 μL/s to about 100 μL/s, about 100 μL/s to about 200 μL/s, or about 200 μL/s to about 500 μL/s. It may be for example about 0.5 μL/s, 1 μL/s, 2 μL/s, 5 μL/s, 10 μL/s, 15 μL/s, 20 μL/s, 25 μL/s, 30 μL/s, 35 μL/s, 40 μL/s, 45 μL/s, 50 μL/s, 55 μL/s, 60 μL/s, 65 μL/s, 70 μL/s, 75 μL/s, 80 μL/s, 85 μL/s, 90 μL/s, 95 μL/s, 100 μL/s, 200 μL/s, 300 μL/s, 400 μL/s, 500 μL/s, 600 μL/s, 700 μL/s, 800 μL/s, 900 μL/s, or 1000 μL/s. The spray lateral movement speed relative to the substrate may be greater than about 0.5 mm/s, or greater than about 1 mm/s, 2 mm/s, 5 mm/s, 10 mm/s, 20 mm/s, 50 mm/s, 100 mm/s, 200 mm/s, or 500 mm/s. It may be from about 0.5 mm/s to about 1000 mm/s, or from about 0.5 mm/s to about 1 mm/s, about 0.5 mm/s to about 2 mm/s, about 0.5 mm/s to about 5 mm/s, about 0.5 mm/s to about 10 mm/s, about 0.5 mm/s to about 20 mm/s, about 0.5 mm/s to about 50 mm/s, about 0.5 mm/s to about 100 mm/s, about 0.5 mm/s to about 200 mm/s, about 0.5 mm/s to about 500 mm/s, about 500 mm/s to about 1000 mm/s, about 200 mm/s to about 1000 mm/s, about 100 mm/s to about 1000 mm/s, about 50 mm/s to about 1000 mm/s, about 20 mm/s to about 1000 mm/s, about 10 mm/s to about 1000 mm/s, about 5 mm/s to about 1000 mm/s, about 2 mm/s to about 1000 mm/s, about 1 mm/s to about 1000 mm/s, about 1 mm/s to about 2 mm/s, about 2 mm/s to about 5 mm/s, about 5 mm/s to about 10 mm/s, about 10 mm/s to about 20 mm/s, about 20 mm/s to about 50 mm/s, about 50 mm/s to about 100 mm/s, about 100 mm/s to about 200 mm/s, or about 200 mm/s to about 500 mm/s. It may be for example about 0.5 mm/s, 1 mm/s, 2 mm/s, 5 mm/s, 10 mm/s, 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 200 mm/s, 300 mm/s, 400 mm/s, 500 mm/s, 600 mm/s, 700 mm/s, 800 mm/s, 900 mm/s, or 1000 mm/s. The spray coating may be performed at below 100% relative humidity, or at below 90%, 80%, 70%, 60%, 50%, 40%, 30%, 20%, or 10% relative humidity. It may be performed from about 0% to about 100% relative humidity, or from about 0% to about 90%, about 0% to about 80%, about 0% to about 60%, about 0% to about 40%, about 0% to about 20%, about 80% to about 100%, about 60% to about 100%, about 40% to about 100%, about 20% to about 100%, or about 20% to about 60% relative humidity. It may be performed at about 0%, 10%, 20%, 30%, 40%, 50%, 60%, 70%, 80%, 90%, or 100% relative humidity. The skilled person will be able to select suitable spray coating conditions to afford desirable properties for the deposited film. For example, the dispensing flow rate and spray lateral movement speed may depend on the desired film thickness.

In the case where the depositing is through printing, the printing may be performed with a dispensing flow rate of greater than about 0.5 μL/s, or greater than about 1 μL/s, 2 μL/s, 5 μL/s, 10 μL/s, 20 μL/s, 50 μL/s, 100 μL/s, 200 μL/s, or 500 μL/s. It may be from about 0.5 μL/s to about 1000 μL/s, or from about 0.5 μL/s to about 1 μL/s, about 0.5 μL/s to about 2 μL/s, about 0.5 μL/s to about 5 μL/s, about 0.5 μL/s to about 10 μL/s, about 0.5 μL/s to about 20 μL/s, about 0.5 μL/s to about 50 μL/s, about 0.5 μL/s to about 100 μL/s, about 0.5 μL/s to about 200 μL/s, about 0.5 μL/s to about 500 μL/s, about 500 μL/s to about 1000 μL/s, about 200 μL/s to about 1000 μL/s, about 100 μL/s to about 1000 μL/s, about 50 μL/s to about 1000 μL/s, about 20 μL/s to about 1000 μL/s, about 10 μL/s to about 1000 μL/s, about 5 μL/s to about 1000 μL/s, about 2 μL/s to about 1000 μL/s, about 1 μL/s to about 1000 μL/s, about 1 μL/s to about 2 μL/s, about 2 μL/s to about 5 μL/s, about 5 μL/s to about 10 μL/s, about 10 μL/s to about 20 μL/s, about 20 μL/s to about 50 μL/s, about 50 μL/s to about 100 μL/s, about 100 μL/s to about 200 μL/s, or about 200 μL/s to about 500 μL/s. It may be for example about 0.5 μL/s, 1 μL/s, 2 μL/s, 5 μL/s, 10 μL/s, 15 μL/s, 20 μL/s, 25 μL/s, 30 μL/s, 35 μL/s, 40 μL/s, 45 μL/s, 50 μL/s, 55 μL/s, 60 μL/s, 65 μL/s, 70 μL/s, 75 μL/s, 80 μL/s, 85 μL/s, 90 μL/s, 95 μL/s, 100 μL/s, 200 μL/s, 300 μL/s, 400 μL/s, 500 μL/s, 600 μL/s, 700 μL/s, 800 μL/s, 900 μL/s, or 1000 μL/s. The print speed may be greater than about 0.5 mm/s, or greater than about 1 mm/s, 2 mm/s, 5 mm/s, 10 mm/s, 20 mm/s, 50 mm/s, 100 mm/s, 200 mm/s, or 500 mm/s. It may be from about 0.5 mm/s to about 1000 mm/s, or from about 0.5 mm/s to about 1 mm/s, about 0.5 mm/s to about 2 mm/s, about 0.5 mm/s to about 5 mm/s, about 0.5 mm/s to about 10 mm/s, about 0.5 mm/s to about 20 mm/s, about 0.5 mm/s to about 50 mm/s, about 0.5 mm/s to about 100 mm/s, about 0.5 mm/s to about 200 mm/s, about 0.5 mm/s to about 500 mm/s, about 500 mm/s to about 1000 mm/s, about 200 mm/s to about 1000 mm/s, about 100 mm/s to about 1000 mm/s, about 50 mm/s to about 1000 mm/s, about 20 mm/s to about 1000 mm/s, about 10 mm/s to about 1000 mm/s, about 5 mm/s to about 1000 mm/s, about 2 mm/s to about 1000 mm/s, about 1 mm/s to about 1000 mm/s, about 1 mm/s to about 2 mm/s, about 2 mm/s to about 5 mm/s, about 5 mm/s to about 10 mm/s, about 10 mm/s to about 20 mm/s, about 20 mm/s to about 50 mm/s, about 50 mm/s to about 100 mm/s, about 100 mm/s to about 200 mm/s, or about 200 mm/s to about 500 mm/s. It may be for example about 0.5 mm/s, 1 mm/s, 2 mm/s, 5 mm/s, 10 mm/s, 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 200 mm/s, 300 mm/s, 400 mm/s, 500 mm/s, 600 mm/s, 700 mm/s, 800 mm/s, 900 mm/s, or 1000 mm/s. The printing may be performed at below 100% relative humidity, or at below 90%, 80%, 70%, 60%, 50%, 40%, 30%, 20%, or 10% relative humidity. It may be performed from about 0% to about 100% relative humidity, or from about 0% to about 90%, about 0% to about 80%, about 0% to about 60%, about 0% to about 40%, about 0% to about 20%, about 80% to about 100%, about 60% to about 100%, about 40% to about 100%, about 20% to about 100%, or about 20% to about 60% relative humidity. It may be performed at about 0%, 10%, 20%, 30%, 40%, 50%, 60%, 70%, 80%, 90%, or 100% relative humidity. The skilled person will be able to select suitable printing conditions to afford desirable properties for the deposited film. For example, the dispensing flow rate and print speed may depend on the desired film thickness.

In the case where the depositing is through roller coating, the roller lateral speed may be greater than about 0.5 mm/s, or greater than about 1 mm/s, 2 mm/s, 5 mm/s, 10 mm/s, 20 mm/s, 50 mm/s, 100 mm/s, 200 mm/s, or 500 mm/s. It may be from about 0.5 mm/s to about 1000 mm/s, or from about 0.5 mm/s to about 1 mm/s, about 0.5 mm/s to about 2 mm/s, about 0.5 mm/s to about 5 mm/s, about 0.5 mm/s to about 10 mm/s, about 0.5 mm/s to about 20 mm/s, about 0.5 mm/s to about 50 mm/s, about 0.5 mm/s to about 100 mm/s, about 0.5 mm/s to about 200 mm/s, about 0.5 mm/s to about 500 mm/s, about 500 mm/s to about 1000 mm/s, about 200 mm/s to about 1000 mm/s, about 100 mm/s to about 1000 mm/s, about 50 mm/s to about 1000 mm/s, about 20 mm/s to about 1000 mm/s, about 10 mm/s to about 1000 mm/s, about 5 mm/s to about 1000 mm/s, about 2 mm/s to about 1000 mm/s, about 1 mm/s to about 1000 mm/s, about 1 mm/s to about 2 mm/s, about 2 mm/s to about 5 mm/s, about 5 mm/s to about 10 mm/s, about 10 mm/s to about 20 mm/s, about 20 mm/s to about 50 mm/s, about 50 mm/s to about 100 mm/s, about 100 mm/s to about 200 mm/s, or about 200 mm/s to about 500 mm/s. It may be for example about 0.5 mm/s, 1 mm/s, 2 mm/s, 5 mm/s, 10 mm/s, 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 200 mm/s, 300 mm/s, 400 mm/s, 500 mm/s, 600 mm/s, 700 mm/s, 800 mm/s, 900 mm/s, or 1000 mm/s. The roller coating may be performed at below 100% relative humidity, or at below 90, 80, 70, 60, 50, 40, 30, 20 or 10% relative humidity. It may be performed from about 0% to about 100% relative humidity, or from about 0% to about 90%, about 0% to about 80%, about 0% to about 60%, about 0% to about 40%, about 0% to about 20%, about 80% to about 100%, about 60% to about 100%, about 40% to about 100%, about 20% to about 100%, or about 20% to about 60% relative humidity. It may be performed at about 0%, 10%, 20%, 30%, 40%, 50%, 60%, 70%, 80%, 90%, or 100% relative humidity. The skilled person will be able to select suitable roller coating conditions to afford desirable properties for the deposited film. For example, the roller lateral speed may depend on the desired film thickness.

In the case where the depositing is through drop casting it may be performed at below 100% relative humidity, or at below 90%, 80%, 70%, 60%, 50%, 40%, 30%, 20%, or 10% relative humidity. It may be performed from about 0% to about 100% relative humidity, or from about 0% to about 90%, about 0% to about 80%, about 0% to about 60%, about 0% to about 40%, about 0% to about 20%, about 80% to about 100%, about 60% to about 100%, about 40% to about 100%, about 20% to about 100%, or about 20% to about 60% relative humidity. It may be performed at about 0%, 10%, 20%, 30%, 40%, 50%, 60%, 70%, 80%, 90%, or 100% relative humidity. The thickness of the film may be affected by the total solids concentration in the mixture. For example, a higher total solids concentration mixture may produce a thicker film through drop casting than a lower total solids concentration mixture with comparable deposition conditions. The skilled person will be able to select suitable drop casting conditions to afford desirable properties for the deposited film.

The method may comprise a step of electrospinning the mixture. Solvent, if present in the mixture, is preferably removed (e.g., substantially removed) or its amount in the mixture is preferably reduced during the electrospinning process, and prior to step (B). The mixture may be electrospun by delivering the mixture from a syringe through a needle and onto a collector. The collector may be, for example, a plate or a mandrel. The collector may be coated with a non-stick material, such polytetrafluoroethylene (PTFE). In certain embodiments, the mixture is electrospun onto a polytetrafluoroethylene-coated mandrel.

In the case where the method comprises a step of electrospinning the mixture, the distance between the needle and collector during the electrospinning process may be from about 1 cm to about 50 cm, or it may be from about 1 cm to about 40 cm, about 1 cm to about 30 cm, about 1 cm to about 20 cm, about 5 cm to about 50 cm, about 10 cm to about 50 cm, about 10 cm to about 30 cm, or about 10 cm to about 20 cm. It may be, for example, about 1 cm, 2 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 25 cm, 30 cm, 35 cm, 40 cm, 45 cm, or 50 cm.

In the case where the method comprises a step of electrospinning the mixture, the needle tip voltage during the electrospinning process may be from about +50 kV to about −50 kV, preferably about +20 kV to about −20 kV, or it may be from about +50 kV to about −20 kV, about +50 kV to about −10 kV, about +50 kV to about 0 kV, about +50 kV to about +10 kV, about +20 kV to about −20 kV, about +30 kV to about −30 kV, about 0 kV to about −50 kV, about 0 kV to about −40 kV, about 0 kV to about −30 kV, or about 0 kV to about −20 kV. It may be, for example, about 50 kV, 40 kV, 30 kV, 20 kV, 19 kV, 18 kV, 17 kV, 16 kV, 15 kV, 14 kV, 13 kV, 12 kV, 10 kV, 8 kV, 6 kV, 5 kV, 2 kV, 1 kV, 0 kV, −1 kV, −2 kV, −5 kV, −6 kV, −8 kV, −10 kV, −12 kV, −13 kV, −14 kV, −15 kV, −16 kV, −17 kV, −18 kV, −19 kV, −20 kV, −30 kV, −40 kV, or −50 kV.

In the case where the method comprises a step of electrospinning the mixture, the collector voltage during the electrospinning process may be from about +50 kV to about −50 kV, preferably about +20 kV to about −20 kV, or it may be from about +50 kV to about −20 kV, about +50 kV to about −10 kV, about +50 kV to about 0 kV, about +50 kV to about +10 kV, about +20 kV to about −20 kV, about +30 kV to about −30 kV, about 0 kV to about −50 kV, about 0 kV to about −40 kV, about 0 kV to about −30 kV, or about 0 kV to about −20 kV. It may be, for example, about 50 kv, 40 kv, 30 kv, 20 kv, 19 kv, 18 kV, 17 kV, 16 kv, 15 kV, 14 kv, 13 kV, 12 kv, 10 kV, 8 kV, 6 kV, 5 kV, 2 kV, 1 kV, 0 kV, −1 kV, −2 kV, −5 kV, −6 kV, −8 kV, −10 kV, −12 kV, −13 kV, −14 kV, −15 kV, −16 kV, −17 kV, −18 kV, −19 kV, −20 kV, −30 kV, −40 kV, or −50 kV.

The flow rate of the mixture through the needle during the electrospinning process may be from about 0.05 mL/min to about 10 mL/min, or it may be from about 0.05 mL/min to about 5 mL/min, about 0.05 mL/min to about 2 mL/min, about 0.05 mL/min to about 1 mL/min, about 0.1 mL/min to about 10 mL/min, about 0.1 mL/min to about 5 mL/min, about 0.1 mL/min to about 2 m/min, about 0.1 mL/min to about 1 mL/min, about 0.5 mL/min to about 10 mL/min, about 0.5 mL/min to about 5 mL/min, about 0.5 mL/min to about 2 m/min, or about 0.5 mL/min to about 1 mL/min. It may be, for example, about 0.05 mL/min, 0.1 mL/min, 0.2 m/min, 0.5 mL/min, 1 mL/min, 1.1 mL/min, 1.2 mL/min, 1.3 mL/min, 1.4 mL/min, 1.5 mL/min, 1.6 mL/min, 1.7 mL/min, 1.8 mL/min, 1.9 mL/min, 2 mL/min, 2.5 mL/min, 3 mL/min, 3.5 mL/min, 4 mL/min, 5 mL/min, 6 mL/min, 7 mL/min, 8 m/min, 9 m/min, or 10 m/min.

Although the above electrospinning conditions are described for laboratory scale electrospinning, the skilled person will understand that the electrospinning conditions may be modified to produce large sheets of the hybrid polymeric material on a commercial scale as may be useful if the hybrid polymeric material is to be used, for example, for wound healing applications, such as for a component of a wound patch.

In certain embodiments, the method does not comprise a step of heating a solution of tropoelastin.

Disclosed herein is a tissue scaffold made according to the method as hereinbefore described. The tissue scaffold may be as hereinbefore described. It may be, for example, a vascular graft, a heart valve, nerve guide, surgical patch, or a wound-healing scaffold.

Disclosed herein is the use of the hybrid polymeric material as hereinbefore described in the manufacture of a tissue scaffold. The tissue scaffold may be as hereinbefore described.

Disclosed herein is a method for regenerating tissue in a subject in need thereof, comprising implanting or applying the tissue scaffold as hereinbefore described in or on the subject. In the case where the tissue scaffold is a vascular graft, for example, the tissue scaffold may be implanted into a suitable position in the subject to replace and/or reinforce a section of an artery, vein, capillary or other component of the vascular system of the subject.

The method may comprise a step of administering an agent prior to, during, and/or following implanting or applying the tissue scaffold. Suitable agents may be selected from, for example, cells, anticoagulants, growth factors, cytokines, enzymes, hormones, extracellular matrix materials, vitamins, other small molecules that promote or assist in tissue regeneration, and combinations thereof. The agent may be an anticoagulant. It may be, for example, heparin or fondaparinux.

The method may comprise a step of culturing a cell line on or in the tissue scaffold ex vivo before implanting or applying the tissue scaffold in or on the subject.

### Examples

The examples disclosed herein are discussed to illustrate application of the disclosure and should not be construed as limiting the disclosure in any way.

### Scaffold Fabrication Process

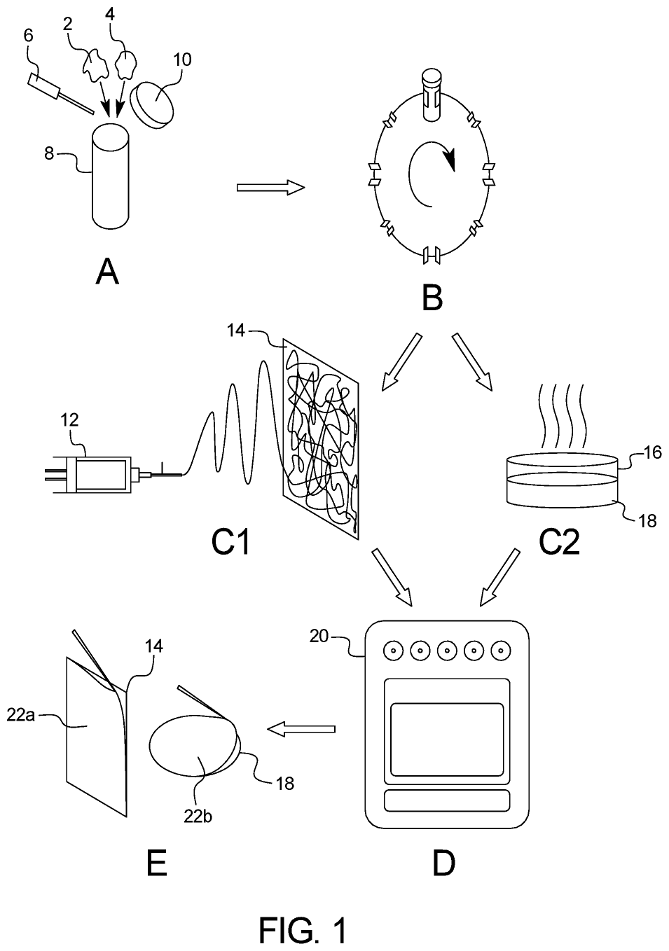

Example schematic scaffold fabrication processes are depicted in FIG. 1. In step A, Tropoelastin (TE) 2 and a polyol-polycarboxylic acid copolymer (polyglycerol sebacate (PGS)) 4 were mixed with hexafluoro-2-propanol (HFP) 6 in container 8 enclosed with lid 10. In step B, the mixture was mixed at 4° C. overnight so that the PGS and TE were completely dissolved in the HFP. The solution was then either transferred to syringe 12 for electrospinning onto substrate 14 (step C1) or transferred to dish 16 for solvent casting onto substrate 18 (step C2). After removal of substantially all the HFP, the materials were transferred to oven 20 for heat curing at 160° C. for 14-18 hours (step D), before the scaffolds (22a, 22b) were removed from the respective substrates (14, 18) (Step E).

TE-PGS mixtures having greater than 30% tropoelastin (by weight compared with the total amount of TE and PGS) were able to be electrospun. A positive voltage of +16 kV and a negative voltage of −16 kV with a tip-to-collector distance of 15 cm were used for all electrospinning processes when PGS was a component of the mixture. 100% tropoelastin scaffolds formed by electrospinning used a positive voltage and a grounded collector. Solvent cast TE-PGS scaffolds could be obtained with any TE-PGS ratios.

### Morphology and 3D Structure of Electrospun TE-PGS Scaffolds

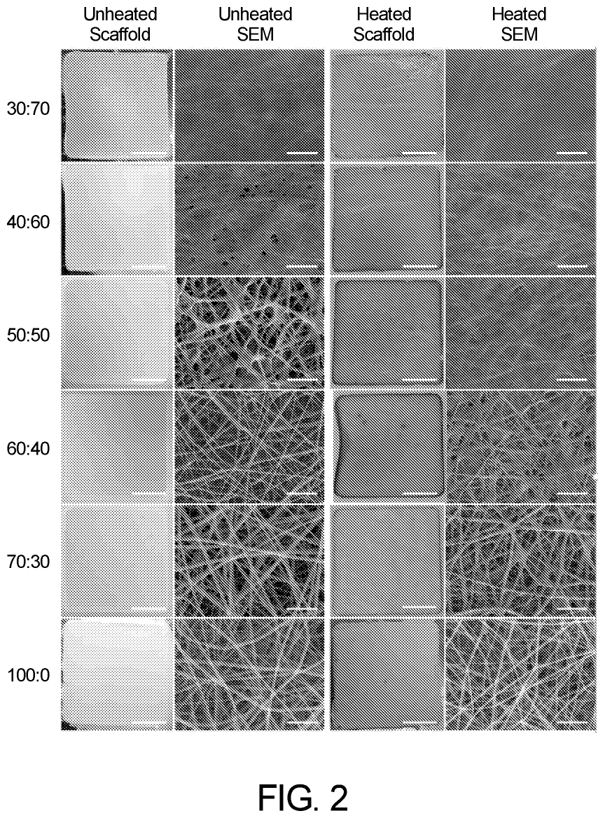

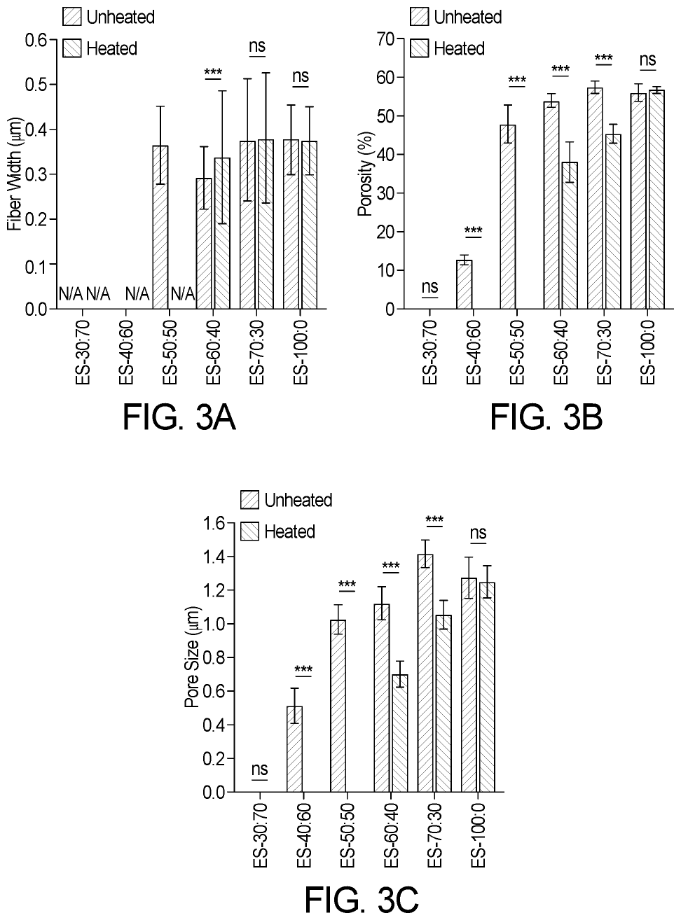

Electrospinning results in scaffolds with a diverse range of microstructures with different fiber width, pore size and porosity. In morphological analysis of unheated scaffolds, PGS tends to spread upon deposition on the collector for TE:PGS-30:70 and TE-PGS-40:60 scaffolds where PGS completely covers the surface. However, the underlying fiber structures can be observed (FIG. 2). Tropoelastin can restrict the spreading of PGS as evidenced by the formation of fibrous morphologies for the TE:PGS-50:50, TE:PGS-60:40 and TE:PGS-70:30 scaffolds, whereby with increasing tropoelastin added, the spreading of PGS becomes less pronounced (FIG. 2). Heating results in the further spreading of PGS, which is seen in the morphology of unheated and heated TE:PGS-50:50 and TE:PGS-60:40 scaffolds (FIG. 2) and can be evidenced by the increase of fiber width and the reduction of the porosity and pore size (FIGS. 3(a), 3(b) and 3(c), and Table 1) for scaffolds having a greater proportion of PGS. Prolonged heating of 16 hours resulted in the formation of scaffolds with stable microstructures.

TABLE 1
Mean value and standard deviation of fiber width, porosity,
and pore size of TE-PGS scaffolds.

| | | | | | | | HeaTro |
|---|---|---|---|---|---|---|---|
| TE-PGS scaffold | | 30:70 | 40:60 | 50:50 | 60:40 | 70:30 | (100:1) |
| Fiber Width | Unheated | N/A | N/A | 0.36 ± 0.09 | 0.29 ± 0.07 | 0.38 ± 0.14 | 0.38 ± 0.08 |
| (μm) | Heated | N/A | N/A | N/A | 0.34 ± 0.15 | 0.39 ± 0.14 | 0.38 ± 0.08 |
| Porosity | Unheated | 0 | 0 | 48.14 ± 4.67 | 54.20 ± 1.67 | 57.69 ± 1.56 | 56.28 ± 2.26 |
| (%) | Heated | 0 | 0 | 0 | 38.31 ± 4.99 | 45.60 ± 2.33 | 56.92 ± 0.84 |
| Pore Size | Unheated | 0 | 0 | 1.01±0.09 | 1.12 ± 0.09 | 1.42 ± 0.08 | 1.27 ± 0.12 |
| (μm) | Heated | 0 | 0 | 0 | 0.70 ± 0.07 | 1.05 ± 0.08 | 1.25 ± 0.10 |

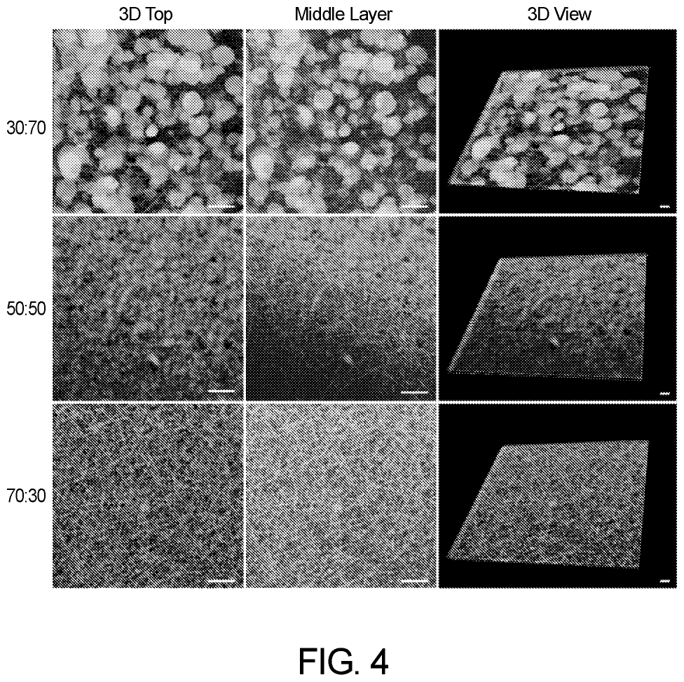

The 3D structures of the scaffolds were visualized by confocal microscopy using the autofluorescence of the scaffolds (FIG. 4). The TE:PGS-30:70 scaffold showed areas with enriched mass of materials that are connected by electrospun fibers. These areas were reduced and finely dispersed in the TE:PGS-50:50 scaffold and supported by underlying fiber structures, forming a fiber-embedded matrix microstructure. The TE:PGS-70:30 scaffold had a completely fibrous microstructure. The skilled person will understand that the diverse range of microstructures with different ratios of the tropoelastin to the polyol-poly carboxylic acid copolymer allows for the use of electrospun ES-PGS scaffolds for a variety of different applications.

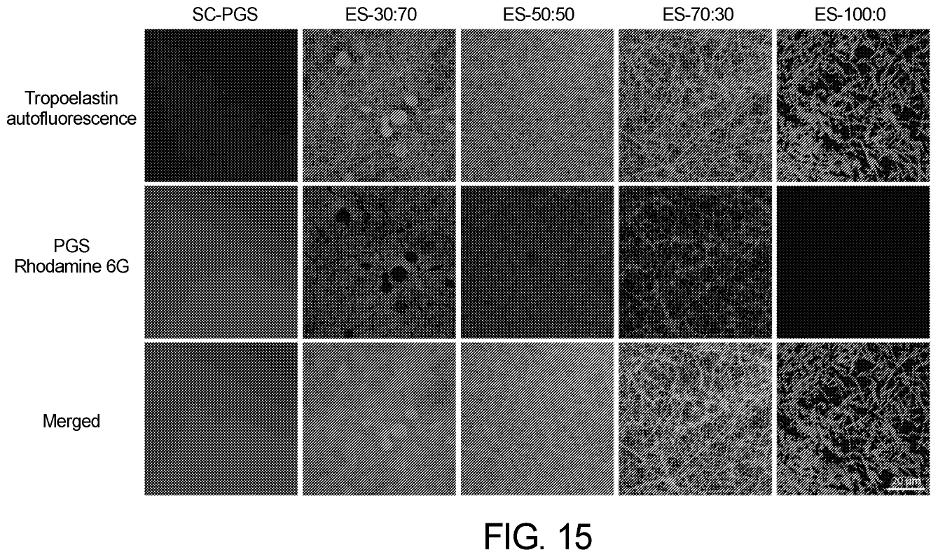

The 3D structures of the scaffolds were also visualized using multiphoton microscopy (FIG. 15). TE is visualized through its autofluorescence and the PGS component is stained by Rhodamine 6G. Solvent cast PGS (SC-PGS) film is used as a control group. SC-PGS has a smooth and homogenous appearance and was stained by Rhodamine 6G. An ES-30:70 scaffold shows areas with enriched masses of TE that are connected by electrospun TE fibers. PGS is not concentrated on the fibers but instead preferentially fills in the spaces between fibers. When increasing TE from 30% to 50%, the TE formed a fine fibrous network without aggregates, which was supported by the underlining PGS matrix to form a fiber-embedded matrix composite. The ES-70:30 scaffold displayed a fibrous microstructure. TE and PGS coexisted on the fiber and a small amount of PGS was concentrated at fiber intersections. ES-100:0 showed the presence of a TE fiber network in the absence of PGS. The diverse range of microstructures allows use of electrospun ES-PGS scaffolds for various applications.

### Fourier-Transform Infrared Spectroscopy-Attenuated Total Reflection

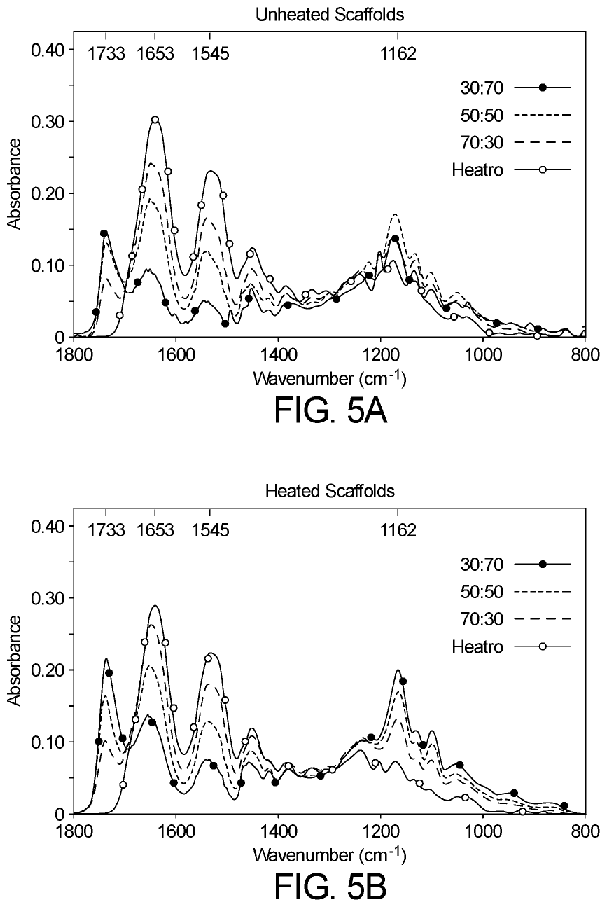

The scaffolds were analysed using FTIR-ATR spectroscopy (FIG. 5). The FTIR-ATR results confirm that there was no chemical change within the scaffolds before and after heating (FIG. 5). Peaks at 1733 cm⁻¹ and 1162 cm⁻¹, which correspond with ester bond and C—O stretching present in PGS, and peaks at 1653 cm⁻¹ and 1545 cm⁻¹, which correspond with Amide I and Amide II of the TE, were observed for all TE-PGS scaffolds. HeaTro (heated 100% tropoelastin) showed no peaks at 1733 cm⁻¹ and 1162 cm⁻¹, and so differed from the TE-PGS scaffolds.

### Swelling in PBS

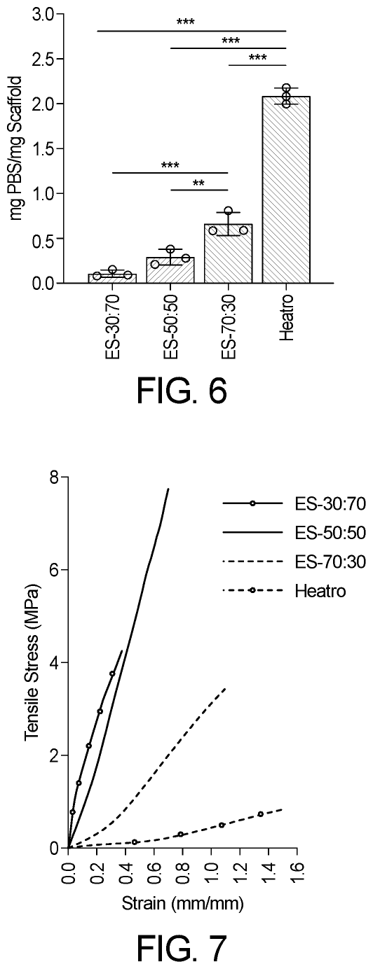

The swelling of the scaffolds was determined in PBS (Table 2). With increasing percentages of tropoelastin, TE-PGS scaffolds swelled more in PBS (FIG. 6 and Table 2), which increased from 0.11 mg PBS/mg scaffold to 0.66 mg PBS/mg scaffold, from ES-30:70 to ES-70:30.

Surprisingly, 100:0 (HeaTro) samples swelled more than 3 times than that of ES-70:30 scaffolds, more than 7 times than that of ES-50:50 scaffolds, and 19 times that of ES-30:70 scaffolds (Table 2); this shows the vastly different behaviour of TE-PGS scaffolds.

TABLE 2
Mean value and standard deviation of scaffold swelling.

| | | | | HeaTro |
|---|---|---|---|---|
| TE-PGS scaffold | 30:70 | 50:50 | 70:30 | (100:0) |
| Swelling (mg PBS/ | 0.11 ± 0.04 | 0.29 ± 0.09 | 0.66 ± 0.13 | 2.09 ± 0.09 |
| mg Scaffold) | | | | |

### Mechanical Properties

The mechanical properties of the scaffolds were determined using tensile testing. The stress-strain curve (FIG. 7) and relevant mechanical properties including ultimate tensile strength, Young's modulus, and elongation at break of TE-PGS scaffolds and 100:0 (HeaTro) were determined (Table 3). With increasing amounts of tropoelastin, TE-PGS scaffolds displayed a decreased Young's modulus and increased elongation, which demonstrated increasing elasticity. Surprisingly, ES-50:50 (TE:PGS-50:50) showed the highest ultimate tensile strength among TE-PGS scaffolds possibly due to its fiber-embedded matrix microstructure where fibers served to reinforce the matrix.

100:0 (HeaTro) samples showed a different stress-strain curve that contrasted with TE-PGS scaffolds with lower ultimate tensile strength and Young's modulus, and higher elongation at failure, demonstrating differences in mechanical behaviour.

TABLE 3
Mean value and standard deviation of ultimate tensile strength, Young’s
modulus and elongation at failure of electrospun TE-PGS scaffolds.

| | | | | HeaTro |
|---|---|---|---|---|
| TE-PGS scaffold | 30:70 | 50:50 | 70:30 | (100:0) |
| Ultimate Tensile | 4.32 ± 0.12 | 8.02 ± 2.14 | 2.76 ± 0.44 | 0.75 ± 0.11 |
| Strength (MPa) | | | | |
| Young’s | 26.21 ± 8.23 | 11.87 ± 6.66 | 1.33 ± 0.14 | 1.16 ± 0.13 |
| Modulus (MPa) | | | | |
| Elongation | 40.68 ± 4.46 | 74.40 ± 9.65 | 105.70 ± 3.90 | 129.30 ± 19.41 |
| at Failure (%) | | | | |

Mass Degradation when Incubated in PBS

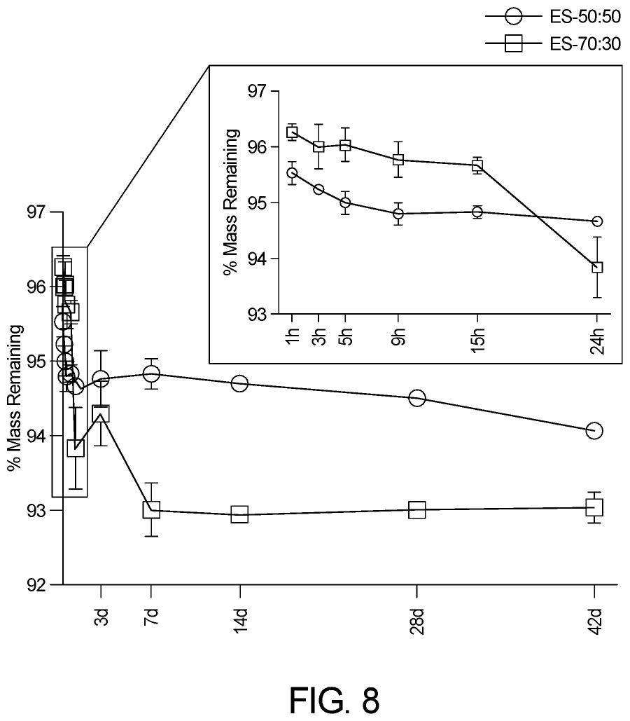

The Scaffolds were incubated in PBS to determine their stability in vitro. ES-50:50 (TE:PGS-50:50) and ES-70:30 (TE:PGS-70:30) lost 5% and 6% of their initial mass during the first day of incubation in PBS (FIG. 8). This loss then slowed. A further 1% mass loss was observed for ES-70:30 within a week. After 1 week, no significant mass loss was seen up to 6 weeks, which confirmed that the TE-PGS scaffolds are very stable in vitro.

Cell Proliferation and Interaction with TE-PGS Scaffolds

TE-PGS scaffolds facilitated improved proliferation of a range of cells-human dermal fibroblasts (HDFs), human umbilical vein endothelial cells (HUVECs) and human coronary artery smooth muscle cells (HCASMCs)—over 7 days compared with PGS scaffolds.

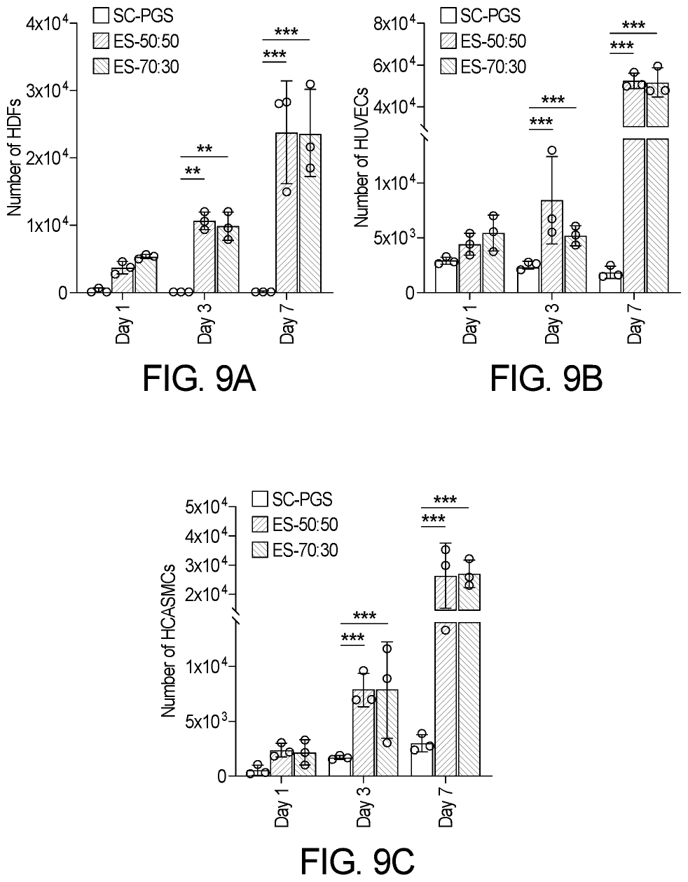

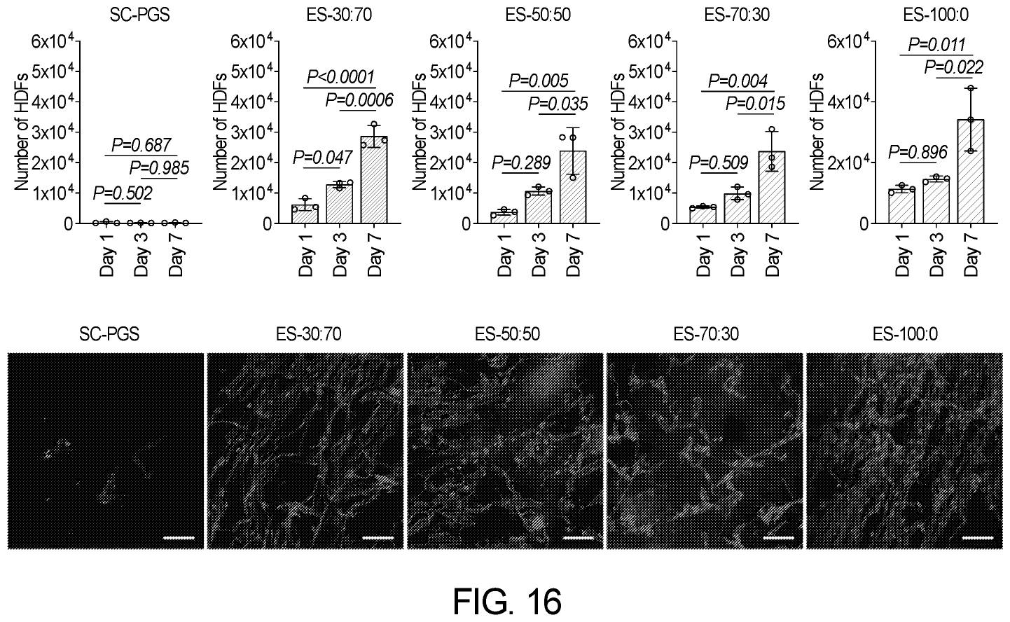

HDFs were cultured on 30:70, 50:50, 70:30, 100:0 TE:PGS electrospun films and SC-PGS films (FIGS. 9A and 16A). Results showed that HDFs proliferate on TE-containing electrospun films up to 7 days, but fail to proliferate on SC-PGS (FIGS. 9A and 16). HDF morphology was studied on day 7 with most of the cells showing elongated morphology on TE-containing films (FIG. 16A). This is compared to HDFs on SC-PGS film that showed reduced number of cells on the film (FIG. 16A) consistent with the results from proliferation assays.

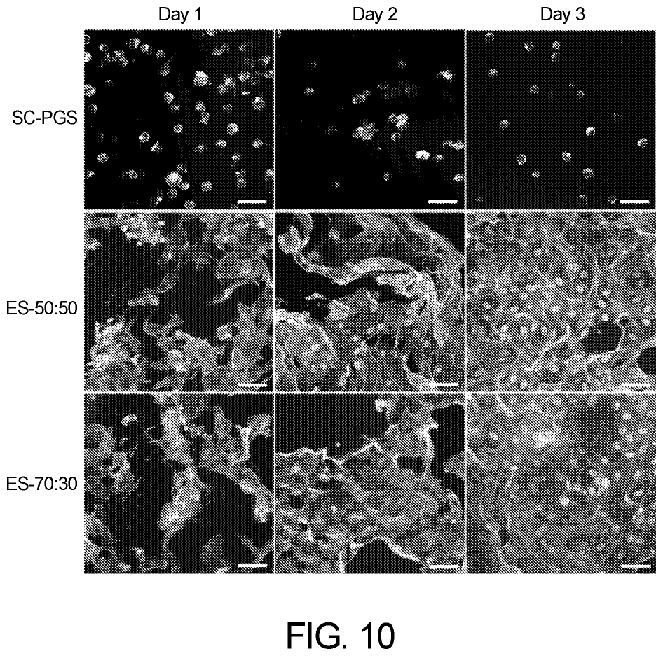

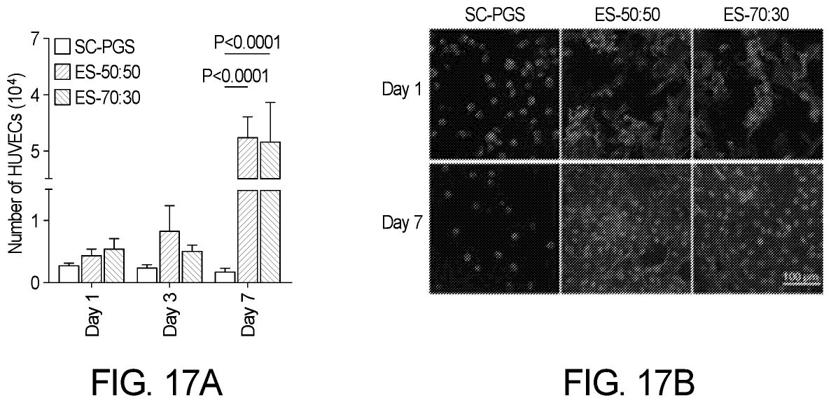

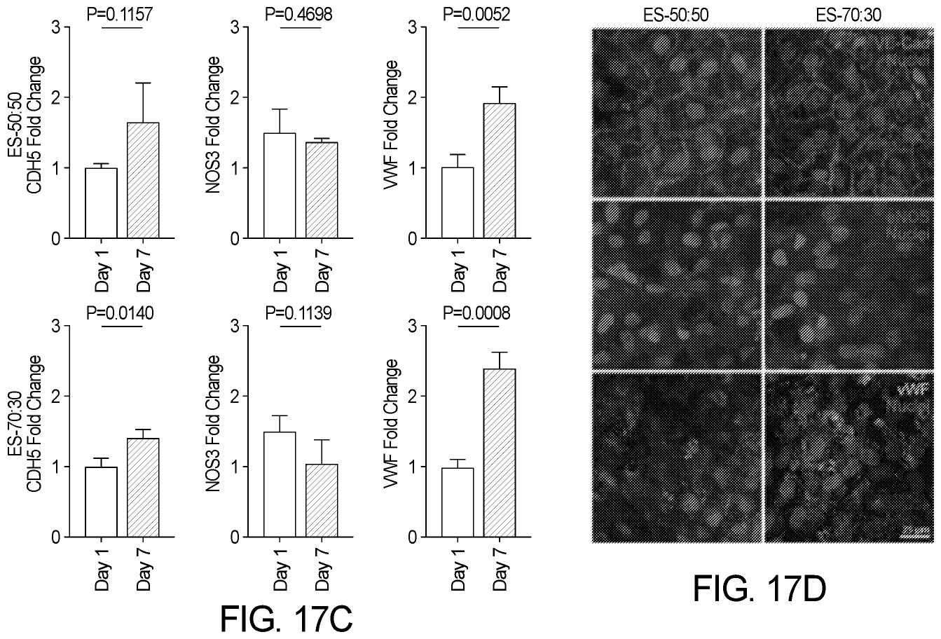

Electrospun TE-PGS scaffolds (both ES-50:50 and ES-70:30) supported HUVEC proliferation (FIGS. 10 and 17A) and near-confluent monolayer formation with a polygonal cellular morphology within 7 days (FIGS. 10 and 17B). This is in contrast with HUVECs that were cultured on SC-PGS, where cells did not proliferate and struggled to survive after 7 days as seen by their rounded morphology. Further analysis showed increased gene expression related to vascular function in HUVECs cultured on the scaffolds from day 1 to day 7, including CDH5 and VWF (FIG. 17C). Vascular-related functional markers are expressed by HUVECs cultured on both ES-50:50 and ES-70:30, including VE-Cadherin, eNOS, and vWF (FIG. 17D).

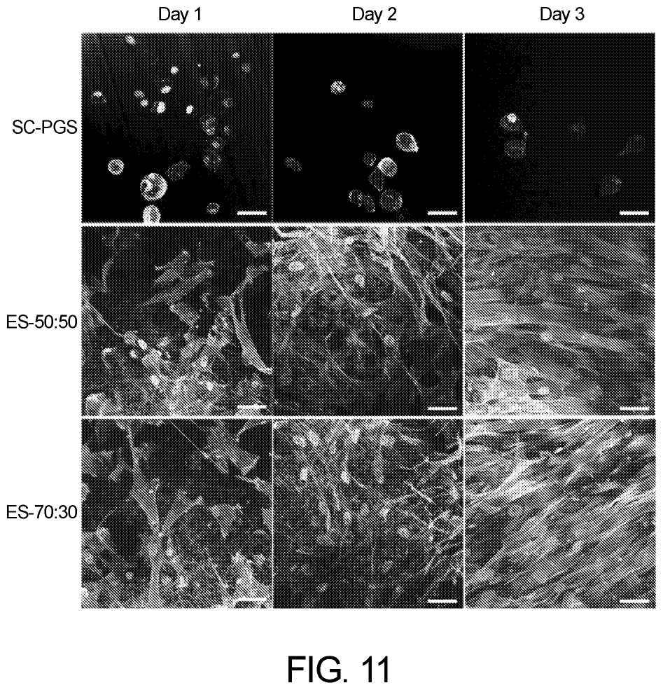

Electrospun TE-PGS scaffolds (Both ES-50:50 and ES-70:30) allowed HCASMCs to proliferate where cell morphology changed from a rhomboid shape on day 1 to a spindle shape on day 7, consistent with a change from synthetic to contractile phenotype (FIG. 11). This is in contrast with HCASMCs culture on PGS where cells did not spread and proliferate even by 7 days as evidenced by their rounded morphology.

### Subcutaneous Implantation—In Vivo Biocompatibility

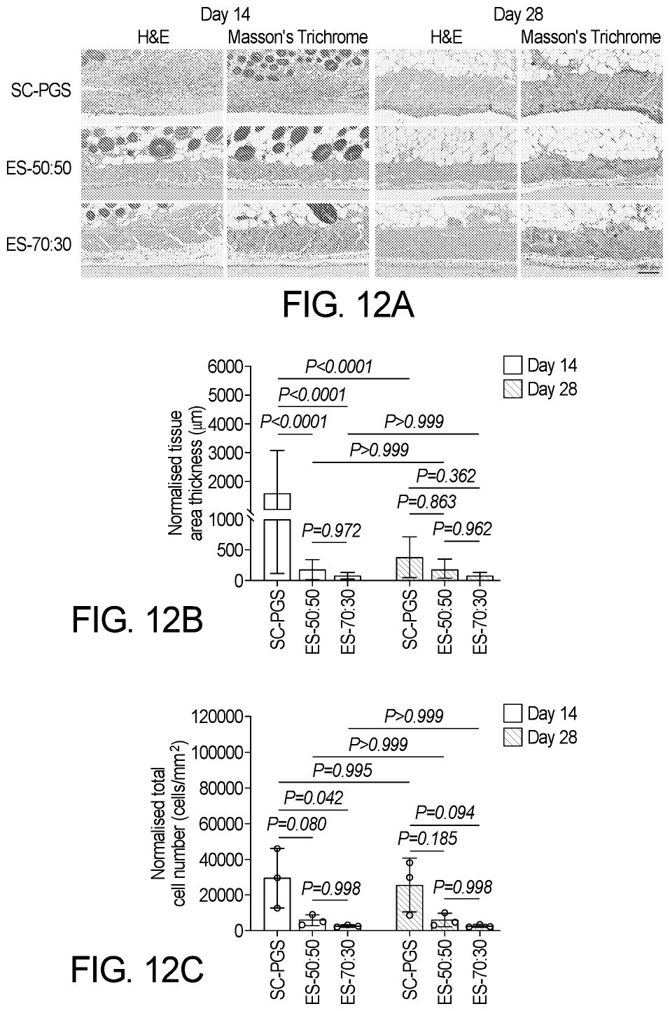

Higher numbers of immune cells were observed for PGS scaffolds subcutaneously implanted in mice at both 2 and 4 weeks compared with TE-PGS scaffolds (FIG. 12A-C). The TE-PGS scaffolds showed a thin fibrous capsule by 2 weeks for ES-50:50 and ES-70:30 (FIG. 12A). The results showed that electrospun TE-PGS scaffolds were well-tolerated in vivo and exhibit less inflammatory responses than PGS.

### Fabrication of Electrospun TE-PGS Vascular Grafts

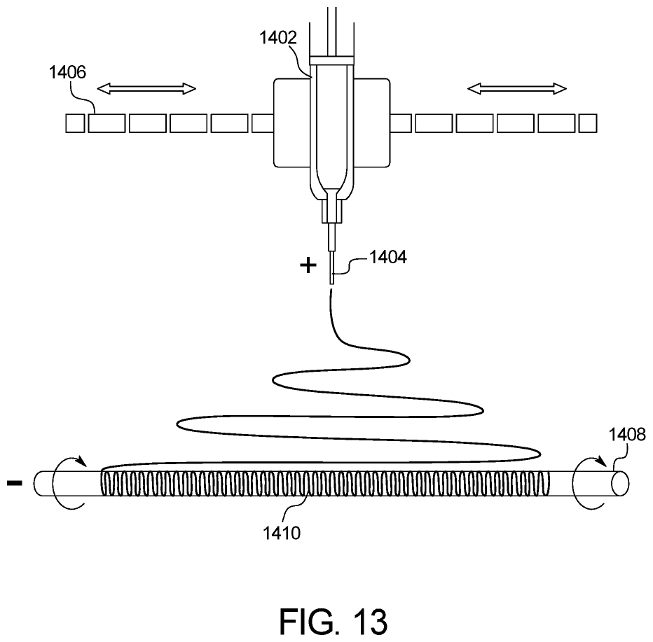

TE-PGS vascular grafts were fabricated by electrospinning onto a rotating mandrel (FIG. 13). Briefly, a Teflon-coated mandrel 1408 with varying diameter sizes was fixed in a shaft holder and rotated using an electric motor at 1000 rpm/min while given a negative charge of from −10 kV to −17 kV. To deliver the TE-PGS, a syringe 1402 was fixed on a band carrier 1406 that moved horizontally, while the needle tip 1404 was connected to a positive charge ranging from +13 kV to +17 kV. A solution composed of 10% (wt/v) TE and 10% (wt/v) PGS in 1 mL HFP was delivered at a rate of 1 mL/hr using a syringe pump. 0.5 mL of this solution was electrospun onto the rotating mandrel, thereby removing substantially all the HFP. The material was then heated at 160° C. for 16 hrs to give the TE-PGS product. The electrospun material could be easily removed from the mandrel both before and after heating due to the Teflon coating.

### Electrospun TE-PGS Vascular Graft Appearance and Morphology

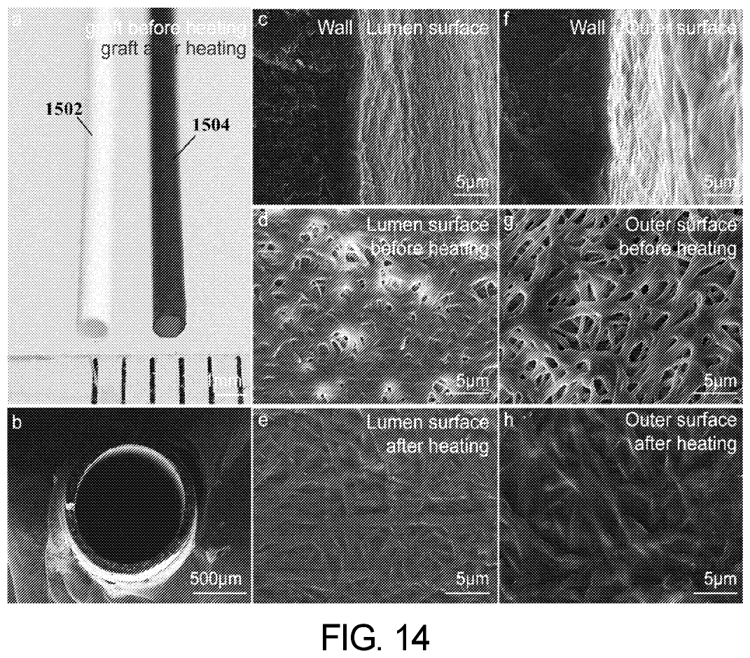

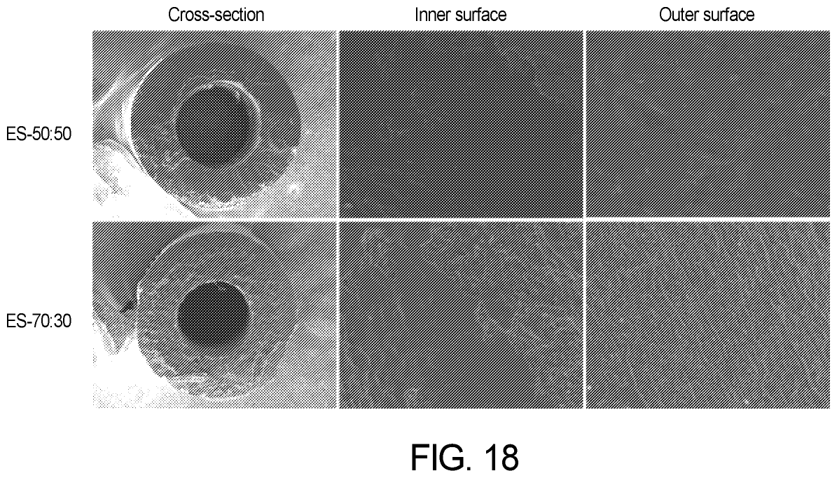

Heating the electrospun material resulted in a change of colour from white to brown, depending on the purity of the source tropoelastin (FIG. 14a). Heated vascular grafts maintained defined geometries with a defined internal diameter and wall thickness (Table 4; FIG. 14b). Heating resulted in the spreading of PGS to form well covered inner and outer surfaces. However, the underlying fiber morphology could still be seen (FIGS. 14c, e, f and h). Thicker grafts can also be fabricated by using more solution, e.g. 0.72 ml of 50:50 or 70:30 ratios (FIG. 18).

TABLE 4
Mean and standard deviation of internal diameter
and wall thickness for heated vascular grafts

| Geometry | Internal diameter | Wall thickness |
|---|---|---|
| Length (μm) | 1062.5 ± 12.4 | 104.1 ± 9.6 |

### Implantation of Electrospun 50:50 Graft in Mouse Aorta

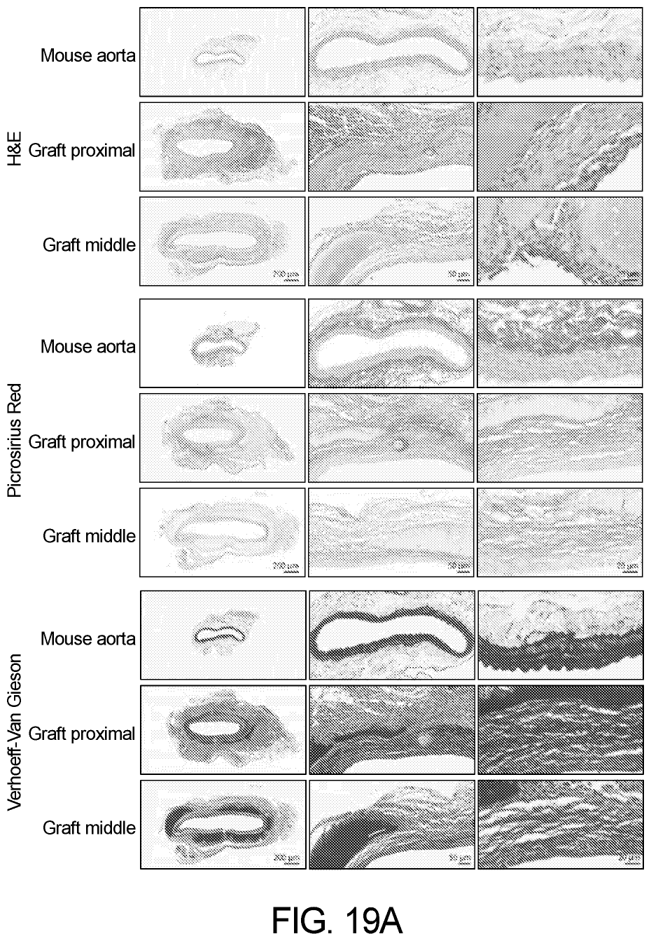

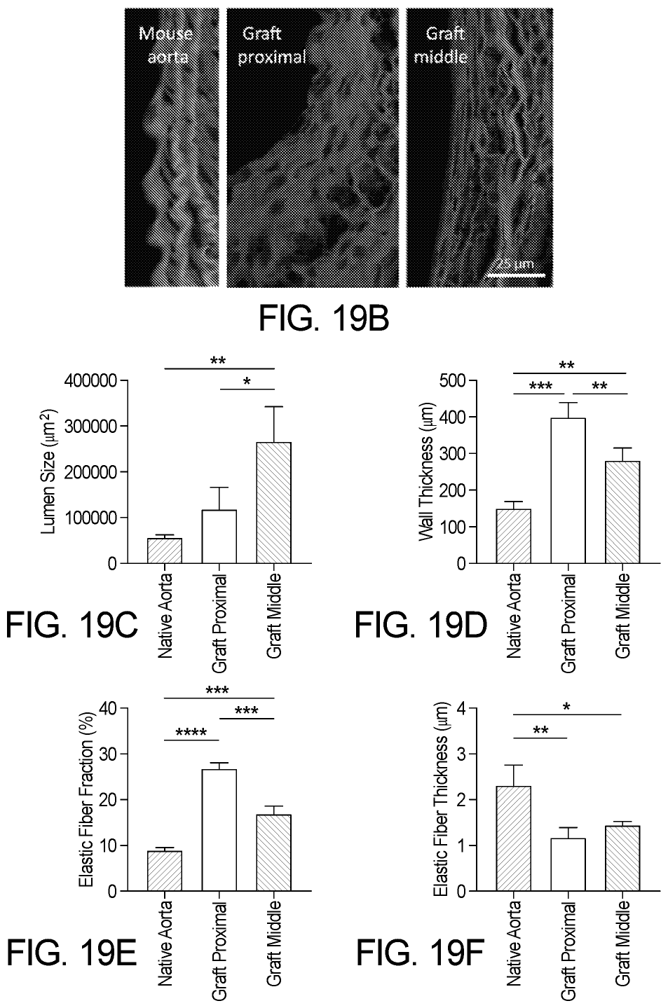

ES-50:50 grafts were used in a standard aorta interposition mouse model and implanted on this basis for 8 weeks at Nationwide Children's Hospital (Sydney, NSW). Analysis of the grafts showed the explanted grafts were partially resorbed over time and showed remodeling with neotissue formation indicated by haematoxylin and eosin (H&E) staining (FIG. 19A, top three rows). Regeneration of collagen is shown at the adventitia of the graft as shown by picrosirius red (PSR) staining (FIG. 19A, middle three rows). Organized, wavy, continuous elastic fibers were found to have regenerated at the intima as shown by Verhoeff-Van Gieson (VVG) staining and elastin autofluorescence (FIG. 19A, bottom three rows, and 19B).

The compliance of the graft slowly increased to the level of the native aorta from week 1 to week 6. Inhomogeneous dilation occurred from week 6 to 8 where the graft middle section was more dilated than the proximal region of the graft (FIG. 19C). This corresponded to a larger wall thickness and a higher fraction of elastic fiber area in the proximal compared to the center of the graft (FIGS. 19D and 19E). By the 8-week mark, regenerated elastic fibers were clearly evident. The amount of total elastin was higher than that in mouse native aorta (FIGS. 19E and 19F).

The skilled person will understand that the new hybrid polymeric material disclosed herein may be suitable for non-tissue scaffold applications, where the mechanical and other properties of the hybrid polymeric material may also be an advantage for such applications. For example, the hybrid polymeric material may be used as a matrix for in vitro experiments involving the growth of cells, such as Caco-2 monolayer experiments for assessing oral bioavailability of new drug candidates.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

1. A hybrid polymeric material comprising:
a tropoelastin; and
a copolymer of a polyol monomer and a polycarboxylic acid monomer.

2. The hybrid polymeric material of claim 1, wherein the polyol monomer is a triol.

3. The hybrid polymeric material of claim 1 or 2, wherein the polyol monomer is glycerol.

4. The hybrid polymeric material of any one of claims 1 to 3, wherein the polycarboxylic acid monomer is a dicarboxylic acid.

5. The hybrid polymeric material of any one of claims 1 to 4, wherein the polycarboxylic acid monomer is a linear C₄-C₂₀ dicarboxylic acid.

6. The hybrid polymeric material of any one of claims 1 to 5, wherein the polycarboxylic acid monomer is sebacic acid.

7. The hybrid polymeric material of any one of claims 1 to 6, wherein the hybrid polymeric material comprises a copolymer of tropoelastin and poly(glycerol sebacate).

8. The hybrid polymeric material of any one of claims 1 to 7, wherein the mass ratio of the tropoelastin to the polyol-polycarboxylic acid copolymer is from about 50:50 to about 70:30.

9. The hybrid polymeric material of any one of claims 1 to 8, wherein the hybrid polymeric material comprises fibers.

10. The hybrid polymeric material of claim 9, wherein the fibers have an average fiber width of from about 200 nm to about 600 nm.

11. The hybrid polymeric material of any one of claims 1 to 10, wherein the hybrid polymeric material has a porous structure.

12. The hybrid polymeric material of claim 11, having an average pore size of from about 0.6 μm to about 1.5 μm.

13. The hybrid polymeric material of claim 11 or claim 12, having a percentage porosity of from about 30% to about 60%.

14. The hybrid polymeric material of any one of claims 1 to 13, wherein the tropoelastin has at least 90% sequence identity with the amino acid sequence of a human tropoelastin isoform across at least 50 consecutive amino acids.

15. The hybrid polymeric material of any one of claims 1 to 14, wherein the tropoelastin has the sequence of a human tropoelastin isoform.

16. A tissue scaffold comprising the hybrid polymeric material of any one of claims 1 to 15.

17. The tissue scaffold of claim 16, having a Young's modulus of from about 1 to about 30 MPa.

18. The tissue scaffold of claim 16 or 17, having an ultimate tensile strength of from about 2 to about 10 MPa.

19. The tissue scaffold of any one of claims 16 to 18, having a percentage elongation at failure of from about 40% to about 110%.

20. The tissue scaffold of any one of claims 16 to 19, where the tissue scaffold loses less than about 10% of its mass when incubated at 37° C. in PBS for 1 week.

21. A method for producing a hybrid polymeric material, said method comprising the following steps:
(A) providing a mixture comprising:
a tropoelastin; and
a copolymer of a polyol monomer and a polycarboxylic acid monomer; and
(B) heating the mixture to form the hybrid polymeric material;
wherein the tropoelastin, polyol monomer, and polycarboxylic acid monomer are as defined in any one of claims 1 to 15.

22. The method of claim 21, wherein the heating is at a temperature of from about 100° C. to about 200° C.

23. The method of claim 21 or 22, wherein the heating is at a temperature of about 160° C.

24. The method of any one of claims 21 to 23, wherein the method is performed at a pressure of about 1 atmosphere.

25. The method of any one of claims 21 to 24, wherein the mixture comprises a solvent, and the method further comprises a step of removing the solvent from the mixture or reducing the amount of solvent in the mixture prior to step (B).

26. The method of claim 25, wherein the solvent is a polar organic solvent, having a boiling point below 80° C.

27. The method of claim 25 or 26, wherein the solvent is hexafluoro-2-propanol.

28. The method of any one of claims 25 to 27, comprising a step of electrospinning the mixture.

29. The method of claim 28, wherein the mixture is electrospun onto a polytetrafluoroethylene-coated mandrel.

30. The method of any one of claims 21 to 29, which does not comprise a step of heating a solution of tropoelastin.

31. A tissue scaffold made according to the method of any one of claims 21 to 30.

32. The tissue scaffold of any one of claims 16 to 20 or 31, which is a vascular graft, a heart valve, nerve guide, surgical patch, or a wound-healing scaffold.

33. Use of the hybrid polymeric material of any one of claims 1 to 15 in the manufacture of a tissue scaffold.

34. A method for regenerating tissue in a subject in need thereof, comprising implanting or applying the tissue scaffold of any one of claims 16 to 20 or 31 to 32 in or on the subject.


SEQUENCE LISTING (46 sequences)

SEQ ID NO: 1 - protein, 5 aa, Artificial Sequence
  Protein 1
GVGVP

SEQ ID NO: 2 - protein, 4 aa, Artificial Sequence
  Protein 2
GGVP

SEQ ID NO: 3 - protein, 6 aa, Artificial Sequence
  Protein 3
GVGVAP

SEQ ID NO: 4 - protein, 9 aa, Artificial Sequence
  Protein 4
AAAKAAKAA

SEQ ID NO: 5 - protein, 18 aa, Artificial Sequence
  Protein 5
GGVPGAIPGGVPGGVFYP

SEQ ID NO: 6 - protein, 9 aa, Artificial Sequence
  Protein 6
GVGLPGVYP

SEQ ID NO: 7 - protein, 7 aa, Artificial Sequence
  Protein 7
GVPLGYP

SEQ ID NO: 8 - protein, 13 aa, Artificial Sequence
  Protein 8
PYTTGKLPYGYGP

SEQ ID NO: 9 - protein, 13 aa, Artificial Sequence
  Protein 9
GGVAGAAGKAGYP

SEQ ID NO: 10 - protein, 10 aa, Artificial Sequence
  Protein 10
TYGVGAGGFP

SEQ ID NO: 11 - protein, 5 aa, Artificial Sequence
  Protein 11
KPLKP

SEQ ID NO: 12 - protein, 12 aa, Artificial Listing
  Protein 12
ADAAAAYKAAKA

SEQ ID NO: 13 - protein, 9 aa, Artificial Sequence
  Protein 13
GAGVKPGKV

SEQ ID NO: 14 - protein, 9 aa, Artificial Sequence
  Protein 14
GAGVKPGKV

SEQ ID NO: 15 - protein, 9 aa, Artificial Listing
  Protein 15
TGAGVKPKA

SEQ ID NO: 16 - protein, 7 aa, Artificial Sequence
  Protein 16
QIKAPKL

SEQ ID NO: 17 - protein, 6 aa, Artificial Sequence
  Protein 17
VAPGVG

SEQ ID NO: 18 - protein, 5 aa, Artificial Sequence
  Protein 18
VPGVG

SEQ ID NO: 19 - protein, 12 aa, Artificial Sequence
  Protein 19
AAAAAAAKAAAK

SEQ ID NO: 20 - protein, 22 aa, Artificial Sequence
  Protein 20
AAAAAAAAAAKAAKYGAAAGLV

SEQ ID NO: 21 - protein, 16 aa, Artificial Listing
  Protein 21
EAAAKAAAKAAKYGAR

SEQ ID NO: 22 - protein, 17 aa, Artificial Sequence
  Protein 22
EAQAAAAAKAAKYGVGT

SEQ ID NO: 23 - protein, 17 aa, Artificial Sequence
  Protein 23
AAAAAKAAAKAAQFGLV

SEQ ID NO: 24 - protein, 29 aa, Artificial Sequence
  Protein 24
GGVAAAAKSAAKVAAKAQLRAAAGLGAGI

SEQ ID NO: 25 - protein, 15 aa, Artificial Sequence
  Protein 25
GALAAAKAAKYGAAV

SEQ ID NO: 26 - protein, 14 aa, Artificial Sequence
  Protein 26
AAAAAAAKAAAKAA

SEQ ID NO: 27 - protein, 12 aa, Artificial Sequence
  Protein 27
AAAAKAAKYGAA

SEQ ID NO: 28 - protein, 11 aa, Artificial Sequence
  Protein 28
CLGKACGRKRK

SEQ ID NO: 29 - protein, 6 aa, Artificial Sequence
  Protein 29
  misc_feature (2)..(2): Xaa can be any naturally occurring amino acid
VXPGVG

SEQ ID NO: 30 - protein, 6 aa, Artificial Sequence
  Protein 30
  misc_feature (2)..(2): Xaa can be any naturally occurring amino acid
ZXPGZG

SEQ ID NO: 31 - protein, 698 aa, Artificial Sequence
  Protein 31
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 32 - protein, 733 aa, Artificial Sequence
  Protein 32
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 33 - protein, 698 aa, Artificial Sequence; identical to SEQ ID NO: 31
  Protein 33

SEQ ID NO: 34 - protein, 660 aa, Artificial Sequence
  Protein 34
GGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 35 - protein, 5 aa, Artificial Sequence
  Protein 35
VPGVG

SEQ ID NO: 36 - protein, 732 aa, Artificial Sequence
  Protein 36
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 37 - protein, 698 aa, Artificial Sequence; identical to SEQ ID NO: 31
  Protein 37

SEQ ID NO: 38 - protein, 660 aa, Artificial Sequence
  Protein 38
MGGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGFFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 39 - protein, 147 aa, Artificial Listing
  Protein 39
SAMGGVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 40 - protein, 200 aa, Artificial Sequence
  Protein 40
SAMGALVGLGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 41 - protein, 60 aa, Artificial Sequence
  Protein 41
GIPPAAAAKAAKYGAAGLGGVLGGAGQFPLGGVAARPGFGLSPIFPGGACLGKACGRKRK

SEQ ID NO: 42 - protein, 47 aa, Artificial Sequence
  Protein 42
GAAGLGGVLGGAGQFPLGGVAARPGFGLSPIFPGGACLGKACGRKRK

SEQ ID NO: 43 - protein, 34 aa, Artificial Sequence
  Protein 43
GADEGVRRSLSPELREGDPSSSQHLPSTPSSPRV

SEQ ID NO: 44 - protein, 34 aa, Artificial Sequence
  Protein 44
GADEGVRRSLSPELREGDPSSSQHLPSTPSSPRF

SEQ ID NO: 45 - protein, 216 aa, Artificial Sequence
  Protein 45
AAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 46 - protein, 183 aa, Artificial Sequence
  Protein 46
AAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK